(19) United States
(12) Patent Application Publication
(10) Pub. No.: US 2026/0321424 A1
(43) Pub. Date: Oct. 15, 2026

(54) DETERMINING A HEALTH-RELATED CONDITION BY SELECTIVELY PERFORMING NUCLEIC ACID SEQUENCING IN RESPONSE TO A SCREENING TEST

(71) Applicant: Foundation Medicine, Inc., Boston, MA (US)

(72) Inventors: Daniel Lieber, Needham, NJ (US); Justin Newberg, Cambridge, MA (US); Xiaotong Yao, Westfield, NJ (US)

(73) Assignee: Foundation Medicine, Inc., Boston, MA (US)

(21) Appl. No.: 19/647,587

(22) Filed: Apr. 14, 2026

Related U.S. Application Data
(60) Provisional application No. 63/789,146, filed on Apr. 15, 2025.

(51) Int. Cl. G16H 50/20; C12Q 1/6806; C12Q 1/6855; C12Q 1/6874; G01N 33/68; G16B 30/00; G16B 40/10; G16B 40/20; G16H 20/10; G16H 50/70

(52) U.S. Cl. CPC G16H 50/20; C12Q 1/6806; C12Q 1/6855; C12Q 1/6874; G01N 33/6854; G01N 33/6887; G01N 33/6893; G16B 30/00; G16B 40/10; G16B 40/20; G16H 20/10; G16H 50/70; G01N 2333/4742; G01N 2333/4748

(57) ABSTRACT

Techniques for identifying a health-related condition based on a protein-based analysis, and optionally based on a nucleic acid sequencing-based analysis are described. In an example method, protein data of a sample obtained from the subject is identified. The sequence read data is indicative of proteins in the sample. The example method further comprises determining a protein score based on the protein data, and determining, based on the protein score, whether to determine a genomic score indicative of sequences of nucleic acid molecules in the sample. In the example method, input features are generated based on the protein score and/or the genomic score and classifying, using a classifier, the health-related condition of the subject based on the input features.

### CROSS-REFERENCE TO RELATED APPLICATION

This application claims priority to U.S. Provisional Application No. 63/789,146, which was filed on Apr. 15, 2025, and is incorporated by reference herein in its entirety.

### BACKGROUND

Many individuals rely on genetic testing to identify whether they have, or are predicted to develop, various health-related conditions. In some cases, single gene testing can be used to assess whether an individual has a particular genetic mutation that is relevant to whether the individual has a genetic disorder or a propensity for disease. Multiple genes, in some cases, can be tested in order to provide even greater context into the individual’s health. Whole exome sequencing (WES) and whole genome sequencing (WGS) can provide even further context.

Significant resources may be utilized to perform genetic testing, due to infrastructure and training requirements as well as testing equipment and reagent costs. Further, extensive genomic sequencing methodologies, such as those utilizing sequence read data obtained by WGS, can result in a substantial amount of data for analysis. For instance, a substantial amount of processing resources may be utilized in order to identify a health-related condition of a subject using sequence read data.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 illustrates an example environment for determining a health-related condition of a subject based on a protein-based analysis, and optionally based on a nucleic acid sequencing-based analysis.

FIG. 2 illustrates an example environment for training and utilizing a predictive model to determine a health-related condition of a subject based on a non-sequencing-based analysis, and optionally based on a sequencing based analysis.

FIG. 3 illustrates an example of training data utilized to train one or more ML models.

FIG. 4 illustrates an example report summarizing predicted categories of a cancer of a subject.

FIG. 5 illustrates an example environment for sequencing various nucleic acid molecules.

FIG. 6 illustrates an example environment illustrating cfDNA, which can be utilized to identify a health-related condition of a subject.

FIG. 7 illustrates an example process for identifying a health-related condition of a subject based on a non-sequencing-based analysis, and optionally based on a sequencing based analysis.

FIG. 8 illustrates one or more devices configured to perform various operations described herein.

### DETAILED DESCRIPTION

Various implementations of the present disclosure relate to techniques for predicting a health-related condition, such as cancer, based on a protein-based analysis, and optionally based on a nucleic acid sequencing-based analysis. In various cases, a sample (e.g., a liquid biopsy sample) is obtained from a subject. The sample, for instance, is divided into a first portion and a second portion. A protein-based analysis is performed on the first portion of the sample. For instance, protein data is generated by identifying and/or quantifying one or more proteins within the first portion of the sample. A preliminary score can be generated based on the protein data. In some cases, the preliminary score is indicative of the likelihood that the subject has the health-related condition, such as one or more types of cancer.

In some examples, nucleic acid molecules are extracted from the sample. For instance, the nucleic acid molecules are extracted from the first portion and/or the second portion of the sample. In some cases, methylation data associated with one or more loci represented by the extracted nucleic acid molecules is generated. The preliminary score can be generated based on the methylation data. In various examples, the preliminary score is indicative of the likelihood that the subject has the health-related condition.

In various cases, the preliminary score is analyzed in order to determine whether to perform an additional, nucleic acid sequencing-based analysis on the second portion of the sample. For instance, if the preliminary score indicates that the subject has the health-related condition, or lacks the health-related condition, with sufficient certainty, then there may be limited diagnostic relevance to performing an additional analysis. However, if the preliminary score indicates that the subject may (or may not) have the health-related condition, then the nucleic acid sequencing-based analysis may be initiated. For instance, sequence read data is generated by sequencing nucleic acid molecules in the second portion of the sample. In various cases, a genomic score is generated based on the sequence read data and is indicative of the likelihood, based on the sequence read data, that the subject has the health-related condition. In some examples, the preliminary score as well as the genomic score may be analyzed to predict the health-related condition. In some examples, the genomic score is analyzed to predict the health-related condition.

Various types of health-related conditions can be predicted using various techniques described herein. In various cases, these techniques are used to determine whether the subject has a cancer type and/or a cancer subtype. In some cases, these techniques are used to determine whether the subject has a non-cancer condition, such as a genetic disorder, an autoimmune disease, cardiac disease, a metabolic disorder, or the like.

Implementations of the present disclosure provide significant improvements to the technical field of cancer diagnosis. Using current technologies, cancer screening tests can include minimally invasive or invasive procedures, such as a Pap smear, or noninvasive procedures that suffer from limitations, such as low sensitivity and potential risks of radiation exposure using medical imaging modalities. Further, the time, cost, and potential side effects (e.g., physical pain) of many cancer screening tests can deter individuals from undergoing the tests. Nucleic acid testing on liquid biopsies has emerged as a potential cancer screening and diagnosis tool, however the cost of performing nucleic acid sequencing is significant, particularly when testing for multiple markers is performed. Performing a low-cost, preliminary protein test can improve accessibility of cancer screening and diagnosis by identifying individuals who can avoid undergoing additional, potentially more costly and/or invasive, testing procedures. Using the techniques described herein, unnecessary use of individuals’ and healthcare system resources can be avoided without sacrificing the accuracy of disease detection.

Various analyses described herein cannot be performed in the human mind, or by pen and paper. For example, the protein data and the sequence read data may represent numerous (e.g., thousands) of residues and bases to be analyzed. In various cases, it would be impossible to manually or mentally identify relevant features based on protein data and/or sequence read data. For instance, it would not be possible to attribute features that are relevant to classification of a particular health-related condition. Particular implementations of the present disclosure are fundamentally tied to computer technology, and do not represent mere automation of processes that are performed manually.

### Example Definitions

As used herein, the terms “deoxyribonucleic acid,” “DNA,” “DNA molecule,” and their equivalents, may refer to a polymer of nucleotides (also referred to as “nucleobases”) containing deoxyribose. The nucleotides in DNA include cytosine (C), guanine (G), adenine (A), and thymine (T). Each DNA nucleotide includes a deoxyribose and a phosphate group. An example single-stranded DNA (ssDNA) molecule includes a chain of covalently bonded DNA nucleotides. In the example ssDNA molecule, the phosphate group of the mth nucleotide is covalently bonded to the deoxyribose of the (m-1)th nucleotide, wherein m is a positive integer greater than 2 and less than or equal to the number of DNA nucleotides in the chain. In various examples, DNA is double-stranded and includes two ssDNA molecules that are complementary to one another and coiled around each other in a double helix form. The nucleotides of one ssDNA molecule are hydrogen bonded to the nucleotides of the other ssDNA molecule. In particular, the pyrimidines (A and T) hydrogen bond to each other, and the purines (C and G) hydrogen bond to each other.

As used herein, the terms “ribonucleic acid,” “RNA,” “RNA molecule,” and their equivalents, may refer to a polymer of nucleotides containing ribose. The nucleotides in RNA include cytosine (C), guanine (G), adenine (A), and uracil (U). Each RNA nucleotide includes a ribose and a phosphate group. In an example RNA molecule, the phosphate group of the nth nucleotide is covalently bonded to the ribose of the (n-1)th nucleotide, wherein n is a positive integer greater than 2 and less than or equal to the number of RNA nucleotides in the chain. Messenger RNA (mRNA) is a type of RNA molecule that is synthesized (or “transcribed”) by RNA polymerase (an enzyme) to be complementary to a gene encoded in a DNA sequence, and is also used by a ribosome to synthesize a polypeptide or protein. An mRNA is therefore an example of a “coding RNA.” In various cases, intron sequences are removed from an mRNA via a process known as “RNA splicing.” MicroRNA (“miRNA”) are single-stranded RNA molecules that perform post-transcriptional gene expression regulation. For instance, a miRNA may bind to a complementary mRNA molecule, thereby cleaving, destabilizing, or otherwise preventing the mRNA molecule from being translated into a polypeptide or protein by a ribosome. In various examples, a miRNA has a length in a range of 21 to 23 RNA nucleotides. As used herein, the terms “non-coding RNA” may refer to a type of RNA that is not translated into a protein. Examples of non-coding RNA include miRNA, transfer RNA (tRNA), and ribosomal RNA (rRNA). The term “functional RNA,” and its equivalents, may refer to any RNA molecule that impacts a biological process. For instance, functional RNA may include mRNA, miRNA, tRNA, rRNA, and the like.

As used herein, the term “base,” and its equivalents, may refer to a monomer of a polymer. For example, a base of DNA or RNA is a nucleotide.

As used herein, the term “base pair,” and its equivalents, may refer to a pair of complementary DNA nucleotides, which are hydrogen-bonded to one another in a double-stranded DNA molecule. For example, a base pair includes a first base in a first ssDNA and a second base in a second ssDNA, wherein the first and second bases are complementary and hydrogen-bonded to one another.

As used herein, the terms “nucleotide,” “nucleobase,” “nucleic acid,” “nucleic acid molecule,” and their equivalents, may refer to an organic molecule that includes a nitrogenous base, a sugar, and a phosphate group. In various cases, a nucleotide is a monomer of DNA or RNA. A nucleotide, for instance, is a chemical structure.

As used herein, the terms “3’ end,” “3-prime end,” and their equivalents, may refer to a terminus of a single-stranded nucleotide polymer that includes a base whose third carbon in its deoxyribose or ribose is bound to a hydroxyl group while being unbound to another base.

As used herein, the terms “5’ end,” “5-prime end,” and their equivalents, may refer to a terminus of a single-stranded nucleotide polymer that includes a base whose fifth carbon in its deoxyribose or ribose ring is unbound to another base. In some cases, the fifth carbon is bound to a phosphate group.

As used herein, the “length” of a polymer refers to a number of covalently bonded monomers that are included in the polymer. For instance, the length of a DNA molecule may be the number of covalently bonded nucleotides in at least one strand of the DNA molecule and/or the number of base pairs in the DNA molecule. In various examples, the length of an RNA molecule may be the number of covalently bonded nucleotides in the RNA molecule.

As used herein, the term “gene,” and its equivalents, refers to a sequence of DNA nucleotides that is transcribed into a functional RNA. The functional RNA, for instance, is RNA that is translated into a polypeptide or protein (e.g., mRNA) or that has some other biological function (e.g., miRNA, tRNA, etc.). A gene is “expressed” when it is used as a template to generate a functional RNA. A subject, for instance, has numerous genes contained in the subject’s genome. A gene may include both introns and exons. As used herein, the term “intron,” and its equivalents, may refer to a subset of DNA nucleotides in a gene that is not used to code for any functional RNA that is expressed by the organism. As used herein, the term “exon,” and its equivalents, may refer to a subset of DNA nucleotides in a gene that is used to code for a functional RNA. For instance, an exon may encode a polypeptide or protein that is expressed by the organism. In various examples, a gene can be represented in data (e.g., as data representative of the sequence of DNA nucleotides in the gene) or as a chemical structure (e.g., as the sequence of DNA nucleotides itself).

As used herein, the term “genome,” and its equivalents, refers to the aggregate of genes of a subject. In various cases, a genome represents the sequences of several linear DNA molecules that are present in a subject’s chromosomes. A “reference genome” refers to an aggregation of genes of one or more reference subjects. In various cases, a genome is represented in data.

As used herein, the term “proteome,” and its equivalents, refers to the aggregate of proteins of a subject. In various cases, a proteome represents the identities or sequences of several polypeptides that are present in a subject. As used herein, the term “peptidome,” and its equivalents, refers to the aggregate of peptides of a subject. For instance, a peptidome represents the identities or the sequences of several peptides (e.g., polypeptides with fewer than 50 amino acids) that are present in a subject.

As used herein, the terms “pangenome,” “pan-genome,” “supragenome,” and their equivalents, refers to an aggregate set of genes from multiple subgroups (e.g., strains) within a population (e.g., a clade) of subjects. A pangenome, for example, indicates genes that are present in all subjects within the population, as well as genes that are present in some of the subjects of the population. A pangenome is represented in data, for instance.

As used herein, the term “transcriptome,” and its equivalents, refers to the aggregate of RNA sequences of a subject. In some cases, a transcriptome is limited to mRNA sequences. In various examples, a transcriptome is represented in data.

As used herein, the term “profile,” and its equivalents may refer to one or more measurements or characteristics of a sample or of a portion of a sample. For instance, a DNA methylation profile of a sample may refer to data indicative of one or more of: a level of methylation (e.g., a quantity of methyl groups) at one or more genetic loci of cells in the sample, a methylation status (e.g., whether the methylation level is above or below a threshold) at one or more genetic loci of cells in the sample, methylation patterns within one or more genetic loci of cells in the sample, a comparison between methylation patterns within the sample (e.g., between different cell types, between tumor and non-tumor cells, etc.), a comparison between a methylation pattern of the sample and a reference methylation pattern, or another metric indicative of DNA methylation of the sample.

As used herein, the term “genomic DNA,” “gDNA,” “chromosomal DNA,” and their equivalents, may refer to DNA molecules that are obtained from a chromosome and/or nucleus of a cell.

As used herein, the terms “DNA fragment,” “fragment,” and their equivalents, may refer to DNA molecules that are excised and/or broken off from a larger DNA molecule.

As used herein, the terms “cell-free DNA,” “cfDNA,” and their equivalents, may refer to DNA fragments that are non-encapsulated and obtained outside of cells within a sample (e.g., a liquid biopsy sample).

As used herein, the terms “circulating tumor DNA,” “ctDNA,” and their equivalents, may refer to a cfDNA molecule that originates from a cancer cell.

As used herein, the terms “end motif,” “terminal sequences,” and their equivalents, may refer to a sequence of nucleotides extending from a 3’ or 5’ end of a DNA or RNA molecule. In various cases, the end motif is shorter than a length of the DNA or RNA molecule. For example, the end motif may have a length in a range of 5 to 30 bases or base pairs, a range of 3 to 30 bases or base pairs, or a range of 1 to 30 base pairs.

As used herein, the term “promoter,” and its equivalents, may refer to a portion of a DNA molecule that binds one or more proteins in order to initiate transcription of a gene. For example, the promotor is located “upstream” of the gene. For example, the promotor is located between the 5’ end of the DNA molecule and the gene. A promotor may include one or more binding sites for RNA polymerase, and/or one or more transcription factor binding sites. In some examples, a promotor includes one or more CpG islands. A promoter, for instance, includes a transcription start site.

As used herein, the terms “CpG island,” “CGI,” “CpG site,” and their equivalents, may refer to a continuous portion of a DNA molecule whose sequence includes greater than a threshold amount (e.g., greater than 50%) of G-C base pairs. In some examples, at least two consecutive G-C base pairs include a cytosine that precedes a guanine (e.g., a 5’-CpG-3’ sequence).

As used herein, the term “enhancer,” and its equivalents, may refer to a portion of a DNA molecule that binds one or more proteins in order to increase the chance that a gene will be transcribed. For instance, an enhancer includes one or more transcription factor binding sites. In various cases, an enhancer includes one or more CpG islands.

As used herein, the term “cancer,” and its equivalents, may refer to a condition of a subject in which particular cells (referred to as “cancer cells”) divide uncontrollably in the subject’s body. In some cases, a cancer is characterized by a location or tissue type from which the cancer cells originated. In some examples, a cancer is characterized by a location or tissue type in which the cancer cells are located.

As used herein, the terms “tumor,” “neoplasm,” and their equivalents, may refer to a mass of tissue including cancer cells.

As used herein, the terms “tissue of origin,” “tissue origin,” and their equivalents, refers to a differentiated type of tissue from which cancer cells in the body of a subject began dividing uncontrollably in the subject’s body.

As used herein, the terms “liquid biopsy,” “fluid biopsy,” and their equivalents, may refer to a process of obtaining a fluid sample from a subject’s body. The sample, for instance, can be referred to as a “liquid biopsy sample.” Examples of fluids that are sampled from the body include blood, plasma, cerebrospinal fluid, sputum, stool, urine, lymphatic fluid, and saliva.

As used herein, the term “tissue biopsy,” and its equivalents, may refer to a process of obtaining a sample of cells from a subject’s body. A tissue biopsy, in various cases, is performed by cutting a mass of cells from the subject’s body. For instance, a tissue biopsy is a procedure performed by a surgeon, interventional radiologist, interventional cardiologist, or other specialized clinician. The term “tissue” or “tissue biopsy sample” can be used to refer to the sample of cells obtained using a tissue biopsy.

As used herein, the term “subject,” and its equivalents, may refer to a human or non-human animal. A subject that is receiving care from at least one care provider may be referred to as a “patient.”

As used herein, the terms “machine learning,” “ML,” “computer learning,” “artificial intelligence,” and their equivalents, may refer to the use of a computing devices to learn patterns in training data. The process of learning these patterns may be referred to as “training.” In particular cases, one or more computing devices may perform machine learning by executing a machine learning model. As used herein, the terms “machine learning model,” “ML model,” and their equivalents, may refer to data encoding instructions that, when executed by at least one computing device, causes the at least one computing device to learn patterns in training data by optimizing one or more metrics, values, or other types of parameters. After training, an ML model, when executed by at least one computing device, causes the at least one computing device to utilize the optimized parameters in order to perform one or more tasks.

As used herein, the term “variant,” and its equivalents, may refer to a difference between a subject genetic sequence and a reference sequence. For instance, a variant may correspond to a difference between one or more nucleotides in a genome of a subject and one or more corresponding nucleotides in at least one reference genome or pangenome. A variant may be characterized by its identity (e.g., what nucleotides are different), its position (e.g., where are the nucleotides located in the genome, what chromosome contains the nucleotides, what gene contains the nucleotides, etc.), its length (e.g., how many nucleotides are different from the reference sequence), its type (e.g., substitution, insertion, deletion, copy number alternation, rearrangement of fusion, etc.), and other features that indicates its significance and/or relevance. In some cases, a variant represents any apparent alteration in a sequence that has been read from a nucleic acid molecule with respect to the reference sequence, such as reads cleaved by restriction enzymes (RE). In various examples, a variant can be represented in data (e.g., by data characterizing the variant) or as a chemical structure (e.g., the nucleotides themselves). As used herein, the term “mutation,” and its equivalents, may refer to a change in a gene.

As used herein, the term “substitution,” and its equivalents, can refer to a nucleotide in a subject sequence that is different than an equivalent nucleotide (e.g., a nucleotide at the same position) in a reference sequence.

As used herein, the term “insertion,” and its equivalents, can refer to a nucleotide in a subject sequence that is added with respect to a reference sequence.

As used herein, the term “deletion,” and its equivalents, can refer to the removal of a nucleotide from a nucleotide sequence.

As used herein, the terms “copy number alternation,” “CNA,” “copy number variation,” “CNV,” and their equivalents, can refer to a portion of a reference sequence that is repeated. In some examples, copy number variation refers to a portion of a reference sequence that is depleted (e.g., repeated less than in a reference genome).

As used herein, the terms “rearrangement of fusion,” “fusion rearrangement,” “translocation,” and their equivalents, can refer to a change in the relative position of one or more portions of a reference sequence, thereby generating a gene that was not present in the reference sequence.

As used herein, the term “sequencing,” and its equivalents, may refer to a process of identifying the order and identity of monomers in a polymer chain, such as the order and identity of nucleotides in a DNA or RNA molecule. The terms “whole genome sequencing,” “WGS,” and their equivalents, may refer to the process of sequencing an entire genome of a subject, including the introns and exons of the genes of the subject. The term “whole exome sequencing,” and its equivalents, may refer to the process of sequencing all exomes of a subject. The term “targeted sequencing,” and its equivalents, may refer to the process of sequencing a portion of the genome of a subject, such as sequencing a single gene of the subject. Various techniques can be utilized to sequence a DNA or RNA molecule, such as massively parallel sequencing (MPS), nanopore sequencing, direct sequencing, Sanger sequencing, or next-generation sequencing. In various cases, sequencing is performed on physical molecules (e.g., RNA or DNA) and is used to generate data. As used herein, the terms “massive parallel sequencing,” “massively parallel sequencing,” “MPS,” and their equivalents, may refer to a technique for simultaneously performing multiple reactions that can be used to identify the order and identity of monomers in multiple polymer chains. In particular cases, massive parallel sequencing can be performed using sequencing-by-synthesis on clonally amplified DNA molecules that are located in spatially separated regions, which are individually monitored by sensors.

As used herein, the term “nanopore sequencing,” and its equivalents, may refer to a technique for identifying the order and identity of monomers in a polymer chain by transporting the polymer chain from a first space to a second space, wherein the first space and the second space are separated by a substrate, by directing the polymer chain through a small hole (known as a “nanopore”) embedded in the substrate, and monitoring a relative electrical signal (e.g., a voltage or current) between the first space and the second space.

As used herein, the terms “non-sequencing-based,” “non-genomic sequencing-based,” and their equivalents may refer to data or techniques that do not include sequencing of nucleic acid molecules. Non-sequencing-based analysis may include, for instance, protein-based analysis, including sequencing-based analysis of proteins. Non-sequencing-based analysis may include, for instance, analysis of nucleic acid molecules that does not include sequencing of the nucleic acid molecules, such as DNA methylation testing, nucleic acid molecule length analysis (e.g., restriction fragment length polymorphism (RFLP) analysis), single nucleotide polymorphism (SNP) profiling assays, polymerase chain reaction (PCR) (e.g., quantitative PCR), methylation-specific PCR (e.g., methylation-specific qPCR), isothermal amplification (e.g., loop-mediated isothermal amplification (LAMP), reverse transcription LAMP, recombinase polymerase amplification (RPA), or nucleic acid sequence-based amplification (NASBA)). While non-sequencing-based data may be indicative of a nucleic acid sequence (e.g., of a mutation in a nucleic acid molecule), determining the non-sequencing-based data does not include techniques to identify the order and identity of monomers in a polymer chain.

As used herein, the term “sensor,” and its equivalents, may refer to a physical device or other apparatus that is configured to detect one or more detection signals.

As used herein, the term “detection signal,” and its equivalents, may refer to a physical signal that can be identified, characterized, or otherwise perceived by a sensor.

As used herein, the term “sequence read data,” and its equivalents, may refer to data that is indicative of an order and identity of monomers in a polymer, such as the order and identity of nucleotides in a DNA or RNA sequence. In various implementations, sequence read data is generated via a sequencing operation.

As used herein, the term “image,” and its equivalents, may refer to 2D or 3D array of data indicative of an array of pixels or voxels.

As used herein, the term “ligating,” and its equivalents, may refer to a process of joining two molecules together, for example, with a chemical bond.

As used herein, the term “adapter,” and its equivalents, may refer to an oligonucleotide that can be ligated to a target nucleic acid molecule. In various cases, an adapter prepares the target nucleic acid molecule for sequencing.

As used herein, the term “bait molecule,” and its equivalents, may refer to a nucleic acid molecule having a region (e.g., a sequence) that is complementary to a region of a target molecule (e.g., cfDNA). A bait molecule includes, for instance, a nucleic acid molecule that can hybridize to (i.e., is complementary to) a target molecule can be used to capture the target molecule. In some instances, the bait molecule is a capture oligonucleotide (or capture probe). In some instances, the bait molecule is suitable for solution phase hybridization to the target molecule. In some instances, the bait molecule is suitable for solid phase hybridization to the target molecule. In some instances, the bait molecule is suitable for both solution-phase and solid-phase hybridization to the target molecule. The design and construction of bait molecules is described in more detail in, e.g., International Patent Application Publication No. WO 2020/236941.

As used herein, the term “amplifying,” and its equivalents, may refer to a process of generating copies of a target molecule, such as a nucleic acid molecule.

As used herein, the term “hybridization,” and its equivalents, may refer to a process by which to complementary single-stranded nucleic acid molecules bind to one another, thereby forming a double-stranded nucleic acid molecule. In certain examples, the double-stranded nature of the nucleic acid molecule is maintained under stringent hybridization conditions. Exemplary stringent hybridization conditions include an overnight incubation at 42 °C in a solution including 50% formamide, 5XSSC (750 mM NaCl, 75 mM trisodium citrate), 50 mM sodium phosphate (pH 7.6), 5XDenhardt's solution, 10% dextran sulfate, and 20 µg/ml denatured, sheared salmon sperm DNA, followed by washing the filters in 0.1XSSC at 50 °C.

As used herein, the term “complementary,” and its equivalents, may refer to a state of two single-stranded nucleic acid molecules with respective sequences that cause the nucleic acid molecules to spontaneously hybridize to one another. One nucleic acid molecule, for instance, may have a sequence that causes each nucleic acid to hydrogen bond to a respective nucleic acid in the other nucleic acid molecule.

As used herein, the terms “therapy,” “treatment,” and their equivalents, may refer to a composition or process that can be used to remediate a health problem. Cancer therapies, for instance, include surgery, radiotherapy, chemotherapy, immunotherapy, cell-based therapies, and the like. Examples of cancer therapies include abemaciclib (Verzenio), abiraterone acetate (Zytiga), acalabrutinib (Calquence), ado- trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), Lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximabcmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecanhziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), and combinations thereof. Examples of cancer therapies also include targeted antibody-based therapies (antibody-drug conjugates, antibody-radioisotope conjugates, and targeted immune cell therapies (e.g., immune effector cells genetically modified to express a chimeric antigen receptor (CAR).

As used herein, the term “treatment-responsive,” and its equivalents, may refer to a type of cancer cells that can be substantially killed using a predetermined type of therapy. For example, cancer cells of a subject may be responsive to a particular treatment if, after the subject is administered the treatment, the cancer cells are diminished by a particular progression level (e.g., radiographic progression level, marker-based progression level, such as prostate-specific antigen (PSA) progression, etc.). Accordingly, the responsiveness of the cells to the type of therapy may indicate the effectiveness of that therapy.

As used herein, the term “treatment-resistant,” and its equivalents, may refer to a type of cancer that cannot be substantially killed using a predetermined type of therapy.

As used herein, the term “metastasis profile,” and its equivalents, may refer to a propensity of a type of cancer to metastasize into one or more differentiated tumor types besides the cancer’s tissue origin. In some implementations, the metastasis profile can further indicate the type of tissue in which the cancer can or is likely to metastasize.

As used herein, the term “clinical trial,” and its equivalents, may refer to a research study used to evaluate a hypothesis based on participation by one or more subjects. In various examples, a clinical trial can be used to assess the efficacy and/or safety of a proposed therapy. A clinical trial may be performed in furtherance of approval of a treatment by a regulatory authority (e.g., the United States Food & Drug Administration (FDA)).

### Description of Example Implementations

Various implementations of the present disclosure will now be described with reference to the accompanying Figures.

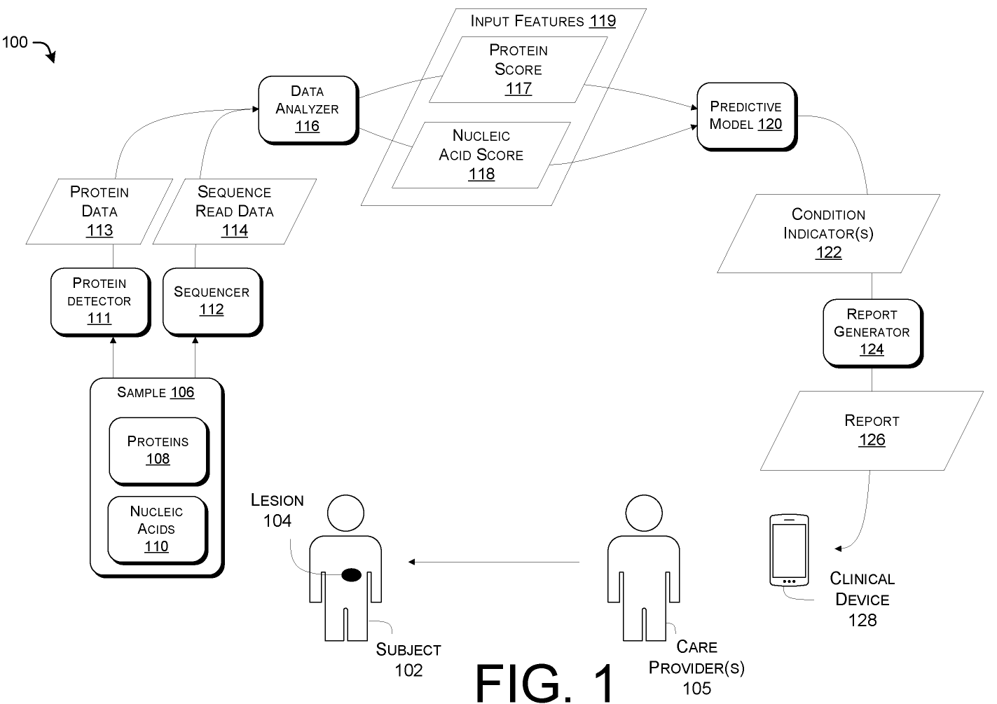

FIG. 1 illustrates an example environment 100 for determining a health-related condition of a subject 102 based on a protein-based analysis, and optionally based on a nucleic acid sequencing-based analysis. In some cases, the subject 102 lacks any apparent disease or other pathological condition. For example, the subject 102 may present to a clinical environment for a medical assessment of the subject 102, such as an evaluation of the general health or well-being of the subject 102. In various cases, the subject 102 presents to the environment 100 as part of a screening assessment for a pathological condition (e.g., cancer). For instance, the subject 102 may schedule an appointment in the environment 100 based on an age or demographic of the subject 102, rather than in response to any symptom or suspected condition. In some examples, the subject 102 may schedule the appointment in the environment 100 in response to identifying they may be at high risk of disease (e.g. based on age, gender, smoking history, body mass index (BMI), family history of disease, environmental exposures, etc.).

In various embodiments, the subject 102 has one or more health-related conditions. For instance, the subject 102 may have one or more types of cancer. The subject 102 may present to the environment 100 with a lesion 104.

In some embodiments, the subject 102 has adrenal cancer, bladder cancer, blood cancer, bone cancer, brain cancer, breast cancer, carcinoma, cervical cancer, colon cancer, colorectal cancer, corpus uterine cancer, ear, nose and throat (ENT) cancer, endometrial cancer, esophageal cancer, gastrointestinal cancer, head and neck cancer, Hodgkin's disease, intestinal cancer, kidney cancer, larynx cancer, leukemia, liver cancer, lymph node cancer, lymphoma, lung cancer, melanoma, mesothelioma, myeloma, nasopharynx cancer, a neuroblastoma, non-Hodgkin's lymphoma, oral cancer, ovarian cancer, pancreatic cancer, penile cancer, pharynx cancer, prostate cancer, rectal cancer, sarcoma, seminoma, skin cancer, stomach cancer, a teratoma, testicular cancer, thyroid cancer, uterine cancer, vaginal cancer, a vascular tumor, or combinations or metastases thereof.

In some embodiments, the subject 102 has B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In various implementations, the subject 102 has a non-cancer condition, such as a genetic disorder, diabetes, cardiac disease, hypertension, a metabolic disorder, a respiratory disease, an infectious disease, an autoimmune disease, a neurological disorder, arthritis, or another health-related condition.

Examples of genetic disorders include Aarskog-Scott syndrome, Aase syndrome, achondroplasia, acrodysostosis, addiction, adreno-leukodystrophy, albinism, ablepharon-macrostomia syndrome, alagille syndrome, alkaptonuria, alpha-1 antitrypsin deficiency, Alport's syndrome, Alzheimer’s disease, amyotrophic lateral sclerosis, asthma, autoimmune polyglandular syndrome, androgen insensitivity syndrome, Angelman syndrome, Apert syndrome, ataxia, ataxia telangiectasia, atherosclerosis, attention deficit hyperactivity disorder (ADHD), autism spectrum disorder, baldness, Batten disease, Becker muscular dystrophy, Beckwith-Wiedemann syndrome, Best disease, bipolar disorder, brachydactyl), Burkitt lymphoma, chronic myeloid leukemia, Charcot-Marie-Tooth disease, Crohn's disease, cleft lip, Cockayne syndrome, Coffin Lowry syndrome, congenital adrenal hyperplasia, Cornelia de Lange syndrome, Costello syndrome, Cowden syndrome, craniofrontonasal dysplasia, Crigler-Najjar syndrome, Creutzfeldt-Jakob disease, cystic fibrosis, deafness, deidocranial dysplasia, depression, diabetes, diastrophic dysplasia, DiGeorge syndrome, Down syndrome, dyslexia, Duchenne muscular dystrophy, Dubowitz syndrome, ectodermal dysplasia, Ellis-van Creveld syndrome, Ehlers-Danlos, epidermolysis bullosa, epilepsy, essential tremor, Fabry disease, facial scapulohumeral dystrophy, familial hypercholesterolemia, familial Mediterranean fever, fragile X syndrome, Friedreich's ataxia, Gaucher disease, glaucoma, glucose galactose malabsorption, glutaricaciduria, gyrate atrophy, Goldberg Shprintzen syndrome, Gorlin syndrome, Hailey-Hailey disease, Haw River syndrome, hemihypertrophy, hemochromatosis, hemophilia, hereditary motor and sensory neuropathy (HMSN), an HLA-B27-associated disorder, Huntington's disease, hypertrophic cardiomyopathy, immunodeficiency with hyper-IgM, Jacobsen syndrome, juvenile onset diabetes, Kennedy’s disease, Klinefelter's syndrome, Kabuki syndrome, Leigh's disease, limb genital syndrome, long QT syndrome, manic depression, Marfan syndrome, Menkes syndrome, miscarriage, mucopolysaccharide disease, multiple endocrine neoplasia, multiple sclerosis, muscular dystrophy, myoclonus epilepsy, myotonic dystrophy, neurofibromatosis, Niemann-Pick disease, Noonan syndrome, obesity, occipital epilepsy syndrome, Parkinson disease, paroxysmal nocturnal hemoglobinuria, Pendred syndrome, peroneal muscular atrophy, phenylketonuria (PKU), polycystic kidney disease, polycystic ovary syndrome, Prader-Willi syndrome, primary biliary cirrhosis, REAR syndrome, Refsum disease, retinitis pigmentosa, retinoblastoma, Rett syndrome, Sanfilippo syndrome, schizophrenia, severe combined immunodeficiency, sickle cell anemia, spina bifida, spinal muscular atrophy, spinocerebellar atrophy, a SRY-associated disorder, sudden adult death syndrome, Tangier disease, Tay-Sachs disease, thalasseaemia, thrombocytopenia absent radius syndrome, Townes-Brocks syndrome, tuberous sclerosis, Turner syndrome, Usher syndrome, von Hippel-Lindau syndrome, Von Willebrand disease, Waardenburg syndrome, Weaver syndrome, Werner syndrome, Williams syndrome, Wilson's disease, xeroderma pigmentosum, Zellweger syndrome, or the like.

Examples of diabetes include type 1 diabetes, type 2 diabetes, gestational diabetes mellitus (GDM), monogenic diabetes, secondary diabetes, or the like.

Examples of cardiac disease include angina, pericarditis, stroke, coronary artery disease, heart valve disease, left ventricular heart failure, right ventricular heart failure, systolic heart failure, diastolic heart failure, ischemic heart disease, an arrhythmia, or the like.

Examples of hypertension include primary hypertension, secondary hypertension, pulmonary hypertension, or the like.

Examples of respiratory diseases include asthma, cystic fibrosis, bronchitis, pleural effusion, pneumonia, bronchiectasis, chronic obstructive pulmonary disease, idiopathic pulmonary fibrosis, acute lung injury, acute respiratory distress syndrome, emphysema, bronchiolitis obliterans or the like.

Examples of metabolic disorders include osteoporosis, hypothyroidism, hyperthyroidism, a mitochondrial disorder, type 2 diabetes, metabolic syndrome, non-alcoholic fatty liver disease, metabolic dysfunction-associated steatotic liver disease (MASLD), alcoholic ketoacidosis, Cushing’s syndrome, hypercalcemia, porphyria, or the like.

Examples of infectious diseases include a viral infection, a bacterial infection, a parasitic infection, a fungal disease, a prion disease, or the like.

Example of viral diseases include or are caused by hepatitis C, norovirus, junin virus, dengue virus, coronavirus, human immunodeficiency virus, herpes simplex, avian flu, chickenpox, cold sores, common cold, glandular fever, influenza, measles, mumps, pharyngitis, pneumonia, rubella, severe acute respiratory syndrome, respiratory syncytial virus, dengue virus, chikungunya virus, lassa virus, ebolavirus, nipah virus, varicella zoster virus, cytomegalovirus (CMV), Epstein-Barr virus (EBV), encephalitis, poliovirus, coxsackievirus, an enterovirus, LaCrosse virus, rabies virus, or the like.

Examples of bacterial disease include diseases caused by E. coli, E. coli STEC, Neisseria gonorrhoeae, Neisseria meningitides, Salmonella enteritidis, Streptococcus pneumoniae, Streptococcus pyogenes, Haemophilus influenza, Klebsiella pneumoniae, Pseudomonas aeruginosa, Staphylococcus aureus, Vibrio cholera, Brucella suis, Mycobacterium tuberculosis, Enterobacter cloacae, Bacillus cereus, Corynebacterium diphtheria, Campylobacter jejuni, Listeria monocytogenes, Campylobacter coli, Bacillus thuringiensis, Yersinia enterocolitica, Helicobacter pylori, Bacteroides fragilis, Clostridium butyricum, Streptococcus agalactiae, Leuconostoc lactis, Shigella sonnei, Zymomonas mobilis, Treponema denticola, Lactobacillus plantarum, Enterococcus faecium, Bacillus liquefaciens, Staphylococcus epidermidis, Serratia marcescens, Citrobacter freundii, Parvimonas micra, Prevotella intermedia, Brucella melitensis, Aeromonas caviae, Clostridium botulinum, Clostridium perfringens, Mycoplasma pneumonia, Salmonella typhimurium, Salmonella paratyphi, Shigella flexnerii, Vibrio parahaemolyticus, Yersinia pseudotuberculosis, Actinobacter baumanii, Bacteroides distasonis, Clostridium leptum, Brevibacterium lactofermentum, Actinobacillus actinobycetemcomitans, Selnomonas ruminatium, Mycoplasma mycoides, Staphylococcus lugdunensis, Lactobacillus rhamnosus, Lactobacillus casei, Lactobacillus acidophilus, Bacillus coagulans, Pyrococcus abyssi, Selenomonas nominantium, Streptococcus fetus, Streptomyces phaechromogenes, Streptomyces ghanaenis, Enterobacter aerogenes, Morganella morganii, Fusobacterium nucleatum, Porphyromonas endodontalis, Porphyromonas gingivalis Nicrococcus luteus, Aeromonas hydrophila, Bacillus anthracis, Bartonella henselae, Bordetella pertussis, Borrelia garinii, Borrelia burgdorferi, Brucella canis, Campylobacter fetus, Clostridium tetani, Francisella tularensis, Legionella pneumophila, Leptospira interrogans, Leptospira santarosai, Leptospira weilii, Mycobacterium leprae, Shigella dysenteriae, Staphylococcus saprophyticus, Streptococcus viridans, Treponema pallidum, Ureaplasma urealyticum, Yersinia pestis, Propionibacterium acnes, or the like.

Examples of fungal diseases include diseases caused by Aspergillus, Bipolaris, Blastomyces, Candida, Cryptococcus, Coccidioides, Curvularia, Exophiala, Histoplasma, Mucorales, Ochroconis, Pseudallescheria, Ramichloridium, Sporothrix, Zygomyctes, Pneumocystis, Trichosporon, or the like.

Examples of parasitic disease include or are caused by Acanthamoeba keratitis, Amoebiasis, Ascariasis, Babesiosis, Balantidiasis, Baylisascariasis, Chagas disease, Clonorchiasis, Cochliomyia, Cryptosporidiosis, Diphyllobothriasis, Dracunculiasis, Echinococcosis, Elephantiasis, Enterobiasis, Fascioliasis, Fasciolopsiasis, Filariasis, Giardiasis, Gnathostomiasis, Hymenolepiasis, Isosporiasis, Katayama fever, Leishmaniasis, Lyme disease, Malaria, Metagonimiasis, Myiasis, Onchocerciasis, Pediculosis, Scabies, Schistosomiasis, Sleeping sickness, Strongyloidiasis, Taeniasis, Toxocariasis, Toxoplasmosis, Trichinosis, Trichuriasis, trypanosomiasis, or the like.

Examples of prion diseases include Creutzfeldt-Jakob disease (CJD), Fatal familial insomnia, Kuru, Gerstmann-Strassler-Scheinker Disease, or the like.

Examples of autoimmune diseases include atopic disease, paraneoplastic autoimmune diseases, arthritis, rheumatoid arthritis, juvenile ankylosing spondylitis, juvenile Reiter's Syndrome, seronegativity, enthesopathy, arthropathy (SEA) Syndrome, dermatomyositis, scleroderma, lupus, systemic lupus erythematosus (SLE), vasculitis, ankylosing spondylitis, Reiter's Syndrome, myolitis, polymyolitis, dermatomyolitis, polyarteritis nodossa, Wegener's granulomatosis, polymyalgia rheumatica, sarcoidosis, sclerosis, primary biliary sclerosis, sclerosing cholangitis, Sjogren's syndrome, psoriasis, plaque psoriasis, guttate psoriasis, inverse psoriasis, pustular psoriasis, erythrodermic psoriasis, dermatitis, atopic dermatitis, atherosclerosis, Still's disease, myasthenia gravis, inflammatory bowel disease (IBD), Crohn's disease, ulcerative colitis, celiac disease, multiple sclerosis (MS), asthma, COPD, rhinosinusitis, eosinophilic esophagitis, eosinophilic bronchitis, Guillain-Barre disease, Type I diabetes mellitus, thyroiditis, Addison's disease, Raynaud's phenomenon, autoimmune hepatitis, graft-versus-host disease (GVHD), transplantation rejection, autoimmune kidney disease, pernicious anemia, alopecia areata, Graves’ disease, Hashimoto Thyroiditis, vitiligo, inflammation, or the like.

Examples of neurological disorders include dementia, Alzheimer’s disease, vascular dementia, Lewy body dementia, frontotemporal dementia, Parkinson’s disease, multiple sclerosis (MS), epilepsy, stroke, migraine, amyotrophic lateral sclerosis (ALS), Huntington’s disease, Guillain-Barré syndrome (GBS), myasthenia gravis, Bell’s palsy, cerebral palsy, neurofibromatosis, myalgic encephalomyelitis, autism spectrum disorder, attention deficit hyperactivity disorder (ADHD), chronic inflammatory demyelinating polyneuropathy (CIDP), muscular dystrophy, or the like.

Examples of arthritis include osteoarthritis, rheumatoid arthritis, psoriatic arthritis, gout, ankylosing spondylitis, juvenile arthritis, septic arthritis, Still's disease, or the like.

In various cases, one or more care providers 105 (also referred to as “healthcare provider(s)”) is responsible for diagnosing and/or treating the subject 102. The care provider(s) 105 may include one or more of a clinician, a surgeon, an anesthesiologist, a pathologist, a laboratory technician, or the like.

According to some implementations, the health-related condition (e.g., cancer) may be initially identified using a noninvasive technique. For example, the lesion 104 may be visualized using an imaging modality, such as ultrasound, x-ray, computed tomography (CT), magnetic resonance imaging (MRI), positron emission tomography (PET), single photon emission CT (SPECT), or any combination thereof.

In various implementations, the care provider(s) 105 are unable to accurately identify the health-related condition of the subject 102 based solely on noninvasive diagnostic techniques. For instance, the care provider(s) 105 cannot conclusively determine a cancer type or subtype of the subject 102. In some implementations, the care provider(s) 105 may not be able to identify an early-stage cancer of the subject 102 due to, for instance, limited resolution of the imaging modality. Further, even noninvasive techniques are inappropriate for screening examinations performed before the subject 102 has any symptoms. For instance, the cost and potential harm (e.g., radiation exposure, in the case of x-ray or CT imaging) of noninvasive techniques outweigh the limited chance of identifying the health-related condition for a population of individuals being evaluated in a pre-disease screening context.

In various implementations, the care provider(s) 105 may be able to identify the health-related condition of the subject 102 by obtaining a sample 106 from the subject 102. In some cases, the sample 106 includes a liquid biopsy sample. The liquid biopsy sample 106, for instance, includes blood, plasma, cerebrospinal fluid, sputum, stool, urine, lymphatic fluid, saliva, or some other fluid obtained from the body of the subject 102. In some cases, a blood sample is obtained intravenously from the subject 102. The liquid biopsy sample 106, according to various examples, is a plasma sample obtained from the blood of the subject 102. The liquid biopsy sample 106, for instance, can be obtained in a minimally invasive procedure, which could be performed by a medical technician rather than a surgeon.

In various cases, the care provider(s) 105 may surgically remove the sample 106 from the lesion 104 to determine genomic information associated with the lesion 104. For instance, the care provider(s) 105 could surgically remove a tissue biopsy sample 106 from the lesion 104. Tissue biopsies, for example, can include endoscopic biopsies, core needle biopsies, fine-needle aspiration, shave biopsies, punch biopsies, incisional biopsies, excisional biopsies, bone marrow biopsies, surgical biopsies, and the like.

The sample 106 includes proteins 108. The proteins 108 may include peptides and/or protein fragments. In some examples, the proteins 108 may include auto-antibodies. The proteins 108 may be located in, or extracted from, cells in the sample 106. In various examples, the proteins 108 are extracted from cells in the sample 106 using mechanical shearing and/or the introduction of a chemical (e.g., a detergent). The proteins 108 may be subsequently isolated from other cellular materials. In various cases, the proteins 108 indicate a full proteome and/or a full peptidome of the sample 106. In some examples, a subset of the proteins 108 may be isolated based on, for instance, size, charge, or affinity.

In various cases, the sample 106 includes nucleic acid molecules 110. According to some examples, the nucleic acid molecules 110 include DNA molecules and RNA molecules. The DNA molecules may include genomic DNA (gDNA). For instance, the nucleic acid molecules 110 include chromosomal DNA that is located in, or extracted from, cells in the sample 106. The RNA molecules may include messenger RNA (mRNA) transcribed from DNA (e.g., genomic DNA (gDNA)) of the subject 102. In some examples, the RNA molecules include at least one of transfer RNA (tRNA), ribosomal RNA (rRNA), or non-coding RNA. Examples of non-coding RNA include microRNA (miRNA), small interfering RNA (siRNA), Piwi-interacting RNA (piRNA), small Cajal body-specific RNA (scaRNA), long intergenic non-coding RNA (lincRNA), circular RNA (circRNA), enhancer RNA (eRNA), natural antisense transcripts (NAT), or the like. Various DNA and/or RNA in the nucleic acid molecules may be indicative of proteins expressed in the cells of the subject 102 and/or the lesion 104.

In some examples, the nucleic acid molecules 110 include DNA molecules and/or RNA molecules that are located in, or extracted from, cells in the sample 106. According to some cases, the DNA molecules and/or the RNA molecules are extracted from nuclei and the cells in the sample 106 using mechanical shearing and/or the introduction of a chemical (e.g., a detergent). The DNA molecules and/or the RNA molecules may be subsequently isolated from proteins and other cellular materials. In some examples, the RNA molecules indicate a full RNA transcriptome of the subject 102 and/or the lesion 104. In various cases, the DNA molecules indicates an entire genome of the subject 102 and/or the lesion 104. Thus, a genome and/or an RNA transcriptome of the subject 102 and/or the lesion 104 can be determined by sequencing the DNA and/or the RNA in the nucleic acid molecules 110. In various cases, the nucleic acid molecules indicate a whole exome of the subject 102 and/or the lesion 104. According to various implementations, the nucleic acid molecules may include tumor DNA (e.g., DNA from tumor cells), tumor RNA, non-tumor DNA (e.g., DNA from non-tumor cells), or non-tumor RNA.

In various implementations, the nucleic acid molecules 110 cell-free DNA (cfDNA). In examples in which the subject 102 has cancer (e.g., the lesion 104 is a cancerous tumor), the cfDNA, for instance, includes circulating tumor DNA (ctDNA) and non-ctDNA. In cases wherein the lesion 104 is a tumor, cancer cells within the lesion 104 will lyse and release the ctDNA into the bloodstream of the subject 102. These cancer cells, for example, include circulating tumor cells (CTCs). Further, other cells additionally shed non-ctDNA into the bloodstream of the subject.

In various cases, the sample 106 is transported to a location that is remote from the subject 102 for further processing. For example, the sample 106 is removed from the subject 102 in a clinical environment (e.g., a hospital) and is then transported to a remote laboratory for further testing and analysis.

In various cases, a health-related condition of the subject 102 can be identified with high certainty by sequencing and analyzing the nucleic acid molecules 110. For instance, it may be possible to identify whether the subject 102 has a particular type of cancer by performing WES on the nucleic acid molecules 110, and analyzing WES data. However, such nucleic acid-based analyses can be costly and resource-intensive. For instance, there may be substantial costs (e.g., in reagents, devices, and personnel) associated with chemically processing the sample 106 in order to sequence the nucleic acid molecules 110. Moreover, a substantial amount of data may be generated by sequencing the nucleic acid molecules 110, which can be difficult to process.

In various implementations of the present disclosure, a preliminary test for detecting the presence and/or amount of the proteins 108 in the sample 106 is performed in order to determine whether sequencing of the nucleic acid molecules 110 is pertinent for identifying the health-related condition of the subject 102 with sufficient certainty. In various cases, a test for the presence and/or amount of the proteins 108 can be performed at a significantly lower cost than a sequencing-based analysis of the nucleic acid molecules 110. In some cases, the presence and/or amount of the proteins 108 in the sample 106 can provide, with sufficient certainty, enough evidence to infer whether the subject 102 has the health-related condition, or lacks the health-related condition. Accordingly, in some cases, it may be possible to identify whether the subject has the health-related condition without incurring the costs associated with a nucleic acid-based analysis. However, if the protein-based preliminary test is insufficient to identify the presence or absence of the health-related condition with a threshold certainty, then implementations of the present disclosure may include sequencing the nucleic acid molecules and analyzing data indicating the sequences of the nucleic acid molecules in order to identify the presence or absence of the health-related condition with the threshold certainty.

In various implementations, the preliminary test can be performed on a liquid biopsy sample. Relying on tissue biopsy samples for detecting health-related conditions has additional limitations. First, it may be dangerous or otherwise impossible to obtain the tissue sample without significant harm to the subject 102. For instance, it may be impossible to obtain tissue samples from sensitive tissues, such as the brain or spinal cord of the subject 102, without severely harming the subject 102. Second, the tissue sample may not be classifiable using conventional histological techniques, such as conventional immunohistochemical staining and review. Third, it is unlikely that the single care provider 105 would be trained to perform the tissue biopsy (which would be performed by a surgeon), to administer anesthesia to the subject 102 during the tissue biopsy (which would be performed by an anesthesiologist), and the analysis of the tissue biopsy (which would be performed by a trained pathologist), such that the classification would utilize multiple highly trained care providers. Even if the health-related condition was classifiable by these means, the coordinated efforts of these care providers could delay classification of the health-related condition and could cause significant expense to the subject 102. In various examples, the delay in classification could cause significant emotional hardship to the subject 102, who could be prevented from receiving an informed prognosis for weeks. Further, the delay in classification could delay administration of a therapy to the subject 102 in order to treat the health-related condition, which could cause lasting harm to the subject 102, particularly in cases in which the health-related condition is associated with aggressive progression. Various implementations described herein may, in some examples, improve accessibility of screening and early detection programs by utilizing noninvasive sample types and providing the preliminary test at a lower cost than a sequencing-based test.

In some cases, the sample 106 is divided into multiple portions. For example, a first portion of the sample 106 is utilized to perform the preliminary test. In various cases, the second portion of the sample 106 is reserved to perform the nucleic acid sequencing-based analysis. In some examples, the preliminary test and the nucleic acid sequencing-based analysis are performed on the same portion of the sample 106.

In various implementations, a protein detector 111 is configured to detect the presence and/or amount of the proteins 108 in the sample 106 (e.g., the first portion of the sample 106). In some cases, the proteins 108 are extracted from the sample 106. The extraction can be performed by the protein detector 111, by another device, manually (e.g., by a laboratory technician), or any combination thereof. Any appropriate extraction method known to those of ordinary skill in the art can be utilized.

In various cases, the protein detector 111 is configured to perform one or more processes (e.g., chemical reactions) on the sample 106 to prepare the proteins 108 for detection and/or sequencing. For instance, the protein detector 111 may extract the proteins 108 from the sample 106. In some cases, the protein detector 111 may apply a capture agent that is configured to bind to a particular protein 108 in the sample 106. Examples of ligands (e.g., capture agents) include antibodies, antibody binding fragments, an aptamer, a peptide, a protein, an enzyme, a tag, a small molecule, a nanobody, or the like. The capture agent, in various cases, may be linked to a detectable label, such as a fluorescent probe, a colorimetric dye, a quantum dot, a ruthenium-based compound (e.g., tris(bipyridyl)ruthenium(II) complex (Ru(bpy)₃²⁺), an iron-based compound (e.g., iron(III) tris(phenanthroline)), a luminescent probe (e.g., a luminescent phosphor, a phosphorescent probe, a luciferase-based probe, a bioluminescent probe, a chemiluminescent probe, etc.), a chromogenic product, a visible byproduct, or the like. The protein detector 111 may be configured to detect a signal associated with the detectable label to determine the presence and/or the quantity of the particular protein in the sample. The signal may include a fluorescent signal, a colorimetric signal, an absorbance, a refractive index, radioactivity, an electrochemical signal, an electrochemiluminscent signal, or the like. The presence and/or level of one or more target proteins in the proteins 108 can be identified by the protein detector 111 based on an analysis of the signal.

The protein detector 111, in some cases, includes a photodetector configured to detect an intensity of light emitted by the detectable label. In various examples, the intensity of the light emitted by the detectable label is proportional to the amount of the particular protein in the sample. In some instances, the protein detector 111 is configured to determine the amount of the particular protein in the sample based on the intensity of the light detected by the photodetector. Examples of photodetectors include photodiodes, photomultiplier tubes (PMTs), charge-coupled devices (CCDs), or complementary metal-oxide semiconductor (CMOS) sensors). In some cases, the protein detector 111 includes a surface plasmon resonance (SPR) sensor, a quartz crystal microbalance (QCM) sensor, an electrochemical sensor, an optical waveguide sensor, or the like configured to detect the detectable label. In various examples, the protein detector 111 may be configured to image at least a portion of the sample 106 to identify the presence of one or more proteins and/or peptides. For example, the protein detector 111 may use fluorescence microscopy to detect a fluorescent probe linked to the capture agent in the sample 106.

In some examples, an individual (e.g., the subject 102, the care provider(s) 105, etc.) can visually identify the presence of the detectable label. For instance, the protein detector 111 may include a lateral-flow based test. The capture agent may be immobilized on a membrane of the test. In some examples, the individual or the protein detector 111 applies the sample 106 (e.g., the first portion of the sample 106) to the test, causing the particular protein to bind to the capture agent. In various cases, the detectable label includes a nanoparticle, a colorimetric dye, a visible byproduct (e.g., of an enzymatic reaction), or the like. The individual may be able to visually identify the presence and/or a level (e.g., by evaluating a color intensity) of the detectable label in the test. In various cases, the individual may identify the presence and/or the level of the detectable label in a liquid test solution (e.g., in a tube, a cuvette, or the like). For instance, the liquid biopsy sample 106 and the detectable label may be added to a tube that includes the capture agent. Based on the interaction between the particular protein and the detectable label, the individual may identify, for instance, a color change corresponding to the presence and/or the level of the particular protein in the sample 106.

Optionally, the protein detector 111 is configured to sequence the proteins 108 in the sample 106. The protein detector 111 may digest the proteins 108 using, for instance, an enzyme. In some examples, the protein detector 111 may ionize the digested proteins. For example, the protein detector 111 may use electrospray ionization (ESI), matrix assisted laser desorption ionization (MALDI), atmospheric pressure chemical ionization (APCI), chemical ionization (CI), electron impact (EI), or the like to ionize the digested proteins. Based on ionizing the digested proteins, the sequencer may determine a mass-to-charge ratio of the digested proteins. In various cases, the protein detector 111 may fragment the digested proteins, such as by collision-induced dissociation, electron transfer dissociation, or the like. The protein detector 111 may be configured to determine the presence and/or amount of the proteins 108 by analyzing the mass-to-charge ratios of the digested protein and the ions associated with the fragmented proteins. For instance, the protein detector 111 may compare the mass-to-charge ratios of the digested protein and the ions associated with the fragmented proteins to those of known target proteins to determine sequences of the proteins 108 in the sample 106.

The protein detector 111 may be configured to detect the presence and/or absence of one or more predetermined target proteins-of-interest in the sample 106. For instance, the protein detector 111 may be configured to detect a presence and/or amount of one or more of: CYFRA, CEA, HE4, CEA, Ferritin, or anti-p53 antibodies. In some examples, the protein detector 111 is configured to detect one or more of: ACE2, ACP5, ACY1, ADA2, ADAM8, ADGRE2, ADGRE5, ADGRG1, AFP, AGR2, ALCAM, ALK, ALPP, ANG, ANG2, ANGPTL4, anti-p53 antibodies, ANXA10, APEX1, AR, AREG, ARSA, B2M, B4GALT1, BAMBI, BMP6, BST2, BTA, BTN2A1, C19ORF12, CA125, CA12, CA15-3, CA19-9, CA242, CA50, CA72.4, CCL14, CCL15, CCL21, CCL22, CD163, CD200, CD200R1, CD22D, CD27, CD274D, CD28, CD300C, CD302, CD38D, CD48, CD6, CD70, CD74, CD79BD, CD83, CDCP1, CDH2, CDH6, CDHR2, CEA, CEACAM21, CEACAM5, CHI3L1, CLEC14A, CLEC5A, CLSTN2, CNDP1, CNTN5, COL4A1D, CRELD2, CRIM1, CRLF1, CRP, CRTAM, CSF1, CTSD, CTSL, CXADR, CXCL13, CXCL17, CXCL9, CYFRA, DCTPP1, DDR1, DKK3, DPP10, DSC2, EFEMP1, EFNA4, EGFL7, ENG, ENPP2, EPHA2D, EPHB4D, EPS8L2, ERBB2D, ESM1, EZR, FABP1, FCRL1, FCRL2, FCRL3, FCRL5, FCRLB, Ferritin, FGFR2B, FLT3LG, FOLR1, FRZB, FSTL3, FUT3, FUT5, FUT5, GAL, GALNT3, Gastrin 17, GCNT1, GDF15, GDNF, GFAP, GGH, GGT1, Glycodelin, GOLM2, GP2, GRN, HAVCR1, HE4, HLA-E, HYOU1, ICAM3, IDS, IFNGR1I, IFNLR1, IGF-1, IGFBP3, IGFBP6, IGFBP7, IGSF3, IL10RB, IL12A, IL12B, IL12BD, IL12RB1, IL18BP, IL18R1, IL2RAB, IL4RD, IL5RAD, IL6R, IL6STD, ITGA5, ITGB2D, ITGB7D, ITIH3, ITM2A, JAM, JCHAIN, KLRB1, KRT14, KRT18, KRT19, LAG3, LAIR2, LAYN, LGALS9, LILRB1, LRIG1, LRP11, LTA, LTBP2, LY9, LYPD8, MARCO, MDK, MILR1, MMED, MMP12, MMP7D, MMP9, MSLN, MSR1, NBL1, NECTIN4D, NFASC, NOMO1, NOS1, NOS3, Notum, NRCAM, NRP2, NT5E, Osteopontin, PARP1, PCDH17, PCOLCE, PDCD1D, PDGFC, Pepsinogen I, Pepsinogen II, PDGFRAB, PGFD, PIGR, PIK3AP1, PIVKA-II, PLAUR, PLXNB2, PREB, PRELP, proGRP, Prolactin, PRSS8, PSA, PSIP1, PTPRS, PVR, RARRES2, RBP2, RBP5, REG3A, REG4, RNASET2, ROR1, RTN4R, SCC, SDC1, SDC4, SEMA4D, SEMA7A, SERPINA9, SEZ6L2, SFTPA2, SH2D1A, SIGLEC1, SIGLEC10, SIGLEC6, SLAMF1, SLAMF6, SLAMF7D, SLAMF8, SPARCL1, SPINK1, SPINK4, SPINT1, SPON2, SPP1, SRP14, ST6GAL1, STC2, STFR2, SULF2, SULT2A1, TCL1A, TFF1, TFF2, TFPI2, TGFBI, TGFBR2, THBD, TIMP1, TNF, TNFRSF10, TNFRSF10A, TNFRSF10B, TNFRSF11B, TNFRSF13, TNFRSF13B, TNFRSF13C, TNFRSF14, TNFRSF19, TNFRSF1A, TNFRSF1B, TNFRSF21, TNFRSF4, TNFRSF8, TNFRSF9, TNFSF13, TNFSF13BD, TNFSF13BI, TNR, TPP1, TREML2, VCAM1, VTCN1, WAS, or YKL40.

The protein detector 111 is configured to generate protein data 113 based on the presence and/or amount of one or more target proteins in the sample 106. In some cases, the protein data 113 may indicate a whole proteome and/or a whole peptidome of at least a portion of the sample 106. In some cases, the protein data 113 may indicate sequences of a subset of the proteins and/or peptides in the sample 106. In various examples, the protein data 113 is indicative of a level of one or more proteins in the sample. The level of a protein may be indicative of a presence, a concentration, an amount, or another metric associated with the protein. In some examples, the level of the protein is indicative of a weighted level or a normalized level (e.g., an amount of a particular protein normalized to the total protein in the sample 106) of the protein.

While FIG. 1 describes a protein-based test as the preliminary test, implementations of the present disclosure are not so limited. In various examples, the preliminary test includes a non-sequencing-based analysis of the nucleic acid molecules 110. For instance, the sample 106 may be processed and then divided. For instance, cfDNA and/or ctDNA may be extracted from the sample 106 and divided into two portions. The first portion of the cfDNA and/or ctDNA may be utilized to perform the non-sequencing-based analysis, and, in some instances, the second portion of the cfDNA and/or ctDNA may be utilized to perform the sequencing-based analysis. The non-sequencing-based analysis may be indicative of a DNA methylation profile of the nucleic acid molecules 110. In some examples, the non-sequencing-based analysis may include PCR or quantitative PCR (qPCR). In some examples, the non-sequencing-based analysis may be indicative of a variant associated with the health-related condition of the subject 102. For instance, the non-sequencing-based analysis may include a CRISPR-based test (e.g., leveraging engineered tracrRNAs and on-target DNAs for parallel RNA detection (LEOPARD), Specific High-Sensitivity Enzymatic Reporter UnLOCKing (SHERLOCK), DNA endonuclease-targeted CRISPR trans reporter (DETECTR), or combinatorial arrayed reactions for multiplexed evaluation of nucleic acids (CARMEN)).

In various examples, the results of the non-sequencing-based analysis of the nucleic acid molecules 110 can be analyzed in order to determine whether the health-related condition of the subject 102 can be identified without performing a sequencing-based analysis. The non-sequencing-based analysis of the nucleic acid molecules 110, in various cases, can be performed at a significantly lower cost than a sequencing-based analysis of the nucleic acid molecules 110. Accordingly, it may be possible to identify whether the subject has the health-related condition by performing the non-sequencing-based analysis of the nucleic acid molecules 110, thereby avoiding incurring the costs associated with a sequencing-based analysis of the nucleic acid molecules 110.

A protein-based or methylation-based analysis can provide valuable context into the health of the subject 102. However, some conventional protein-based tests can have limitations, such as inadequate sensitivity (e.g., particularly for early detection), high false-positive rates (e.g., when analyzing a limited number of biomarkers), and limited use in determining stage or progression of disease. Similarly, while non-sequencing-based analyses of the nucleic acid molecules 110 can provide information about the health-related condition, they can suffer from similar limitations as protein-based tests. A false or inadequate diagnosis could cause emotional hardship to the subject 102 and delay appropriate classification and treatment, causing physical harm to the subject 102.

In various implementations, a data analyzer 116 is configured to determine whether the protein data 113 is sufficient evidence to infer whether the subject 102 has at least one health-related condition (e.g., a type of cancer). In some cases, the data analyzer 116 calculates a preliminary score 117 based on the protein data 113. In various cases, the data analyzer 116 includes a model configured to calculate the preliminary score 117 based on the protein data 113. For instance, the data analyzer 116 may include one or more ML models that are pretrained to calculate the preliminary score 117. The ML model(s), in some examples, include a logistic regression model, a random forest model, an artificial neural network (ANN), a decision tree, a k-nearest neighbor (KNN) model, a support vector machine (SVM), or a naïve Bayes classifier. For example, parameters of the ML model(s) may have been previously optimized based on training data including features of individuals within a population omitting the subject 102. For instance, the ML model(s) was trained using an unsupervised or semi-supervised learning technique, wherein the parameters were optimized to categorize (e.g., cluster) the features of the population. In some cases, the ML model(s) was trained using a supervised learning technique and pre-classified data, wherein the training data further included ground truth preliminary scores of the individuals in the population, such that the parameters were optimized to minimize a loss between predicted preliminary scores generated by the ML model(s) based on the features of the population and the ground truth preliminary scores experienced by the individuals in the population. To increase training robustness, the population represented by the training data may include individuals without the health-related condition, as well as individuals with a variety of types of presentations of the health-related condition. In some examples, the data analyzer 116 may include a regression model, threshold rule, confidence interval, or other type of statistical model capable of categorizing the preliminary score 117 based on the protein data 113.

In various cases, the preliminary score 117 is indicative of a likelihood or probability that the subject 102 has the health-related condition or lacks the health-related condition. For instance, the preliminary score 117 associated with the subset of the proteins 108 including CYFRA, CEA, and HE4 may be indicative of a likelihood that the subject 102 has lung cancer. In some cases, the preliminary score 117 indicates the presence and/or amount of one or more proteins-of-interest in the first portion of the sample. The preliminary score 117 may include one or more quantitative values. In various cases, the preliminary score 117 includes a multidimensional output. According to some cases, the data analyzer 116 is configured to compare the preliminary score 117 to one or more threshold(s). The threshold(s) may be determined based clinical guidelines, clinical data (e.g., data obtained from a population of subjects), data obtained from a database, or the like. In various examples, the threshold(s) are determined based on protein levels of a population of subjects with a particular health-related condition and/or a population of subjects without the particular health-related condition. In some cases, upregulation of a first subset of the proteins 108 and downregulation of a second subset of the proteins 108 are indicative of the health-related condition. The preliminary score 117 may be indicative of whether levels of the first subset of the protein 108 are above the corresponding thresholds and whether levels of the second subset of the protein 108 are below the corresponding thresholds.

Based on the comparison, the data analyzer 116 may determine that there is sufficient evidence to infer that the subject 102 has a health-related condition, or lacks the health-related condition, without further analysis. Accordingly, it may be possible to determine whether the subject 102 has the health-related condition without performing costly nucleic acid-based testing. In some cases, the second portion of the sample 106 can be discarded without further testing.

In various examples, the data analyzer 116 identifies one or more follow-up tests for the subject 102. For instance, the preliminary score 117 may indicate that the subject 102 likely has cancer, and the data analyzer 116 may select an imaging procedure to identify the location of the lesion 104. In some examples, the data analyzer 116 selects collecting a tissue biopsy sample from the subject 102. In some examples, the data analyzer 116 may analyze a subset of the nucleic acid molecules 110 in the sample 106 to determine additional information related to the health-related condition. For instance, the data analyzer 116 may identify a subset of the nucleic acid molecules 110 that is associated with a stage or aggressiveness of the health-related condition, a prognosis of the health-related condition, a responsiveness of the subject 102 to a treatment, or the like. The care provider(s) 105 may be able to better characterize the health-related condition of the subject 102 and/or identify a suggested treatment predicted to successfully treat the health-related condition of the subject 102 based on the results of the follow-up test. Accordingly, in some cases, resources (e.g., financial resources, clinical resources, etc.) can be utilized more effectively by performing the preliminary test optionally followed by the follow-up test, rather than performing a nucleic acid sequencing-based analysis.

However, in some examples, the data analyzer 116 determines that the protein data 113 is insufficient to infer the presence of the health-related condition in the subject 102. For example, the data analyzer 116 may determine that the preliminary score 117 is below a threshold. In these circumstances, the data analyzer 116 may determine that further evidence can be utilized to determine whether the subject 102 has the health-related condition, or lacks the health-related condition, with a threshold certainty (e.g., a certainty associated with sufficient diagnostic accuracy for assistance to the care provider 105).

In some implementations, the care provider(s) 105 can identify the health-related condition of the subject 102 based on a sequencing-based analysis of the nucleic acid molecules 110 in the sample 106. Nucleic acid detection can identify disease-related genes and/or proteins with greater sensitivity than protein detection due to, for instance, the ability for amplification. In some examples, nucleic acid biomarkers can provide additional information about a health-related condition, such as, for instance, responsiveness of a tumor to a particular treatment. However, nucleic acid testing can have limited accessibility, due to infrastructure and training needs and high costs. Furthermore, some nucleic acid tests depend on the tumor burden being above a particular threshold. Accordingly, nucleic acid testing may not be appropriate for screening programs in which most participants will test negative and/or for early detection in which participants may have low tumor burdens.

In various implementations, a sequencer 112 is configured to generate sequence read data 114 indicating the sequences of the nucleic acid molecules 110 in the second portion of the sample 106. The sequencer 112, for instance, includes one or more devices that are configured to generate the sequence read data 114 by processing at least a portion of the sample 106. In some cases, the nucleic acid molecules 110 are extracted from the sample 106. The extraction can be performed by the sequencer 112, by another device, manually (e.g., by a laboratory technician), or any combination thereof. Any appropriate extraction method known to those of ordinary skill in the art can be utilized.

In various cases, the sequencer 112 is configured to perform one or more processes (e.g., chemical reactions) on the nucleic acid molecules 110 in order to prepare the nucleic acid molecules 110 for sequencing. For instance, the sequencer 112 may ligate adapters onto the nucleic acid molecules 110 and/or amplify the nucleic acid molecules 110, such that numerous copies of the ligated nucleic acid molecules 110 are available for sequencing. Examples of the adapters include, for example, amplification primers, flow cell adapter sequences, substrate adapter sequences, or sample index sequences. The nucleic acid molecules 110 (e.g., the ligated nucleic acid molecules 110) may be amplified by generating multiple copies of the nucleic acid molecules 110 using one or more techniques such as polymerase chain reaction (PCR), a non-PCR amplification technique, or an isothermal amplification technique. In some cases, the sequencer 112 is configured to perform a comprehensive genomic profiling test, whole exome sequencing (WES), whole exome sequencing (WES) test, a gene expression profiling test, or a cancer hotspot panel test on the nucleic acid molecules 110.

The sequencer 112 may identify the length, position, and identity of the bases in the nucleic acid molecules 110 by sequencing the nucleic acid molecules 110 (e.g., the amplified and/or ligated nucleic acid molecules 110). In various cases, the sequencer 112 is a next-generation sequencer configured to perform next-generation sequencing (NGS) on the nucleic acid molecules 110. In various implementations, the sequencer 112 utilizes first-generation sequencing (e.g., Sanger sequencing), second-generation sequencing (e.g., massive parallel sequencing), third-generation sequencing (e.g., nanopore sequencing), or a combination thereof. In some cases, the sequencer 112 is configured to sequence substantially all of the nucleotides of all of the nucleic acid molecules 110 fragments obtained from the sample 106. In some examples, the sequencer 112 is configured to perform targeted sequencing. For instance, the sequencer 112 may determine whether the nucleic acid molecules 110 fragments contain one or more predetermined sequences at one or more genomic locations.

In various cases, the sequencer 112 includes one or more sensors that are configured to detect physical signals (also referred to as “detection signals”) that are indicative of the nucleotide sequences of the nucleic acid molecules 110. The sequencer 112 may perform sequencing-by-synthesis. For example, the sequencer 112 may include one or more optical sensors configured to detect optical signals emitted from fluorescently tagged nucleotide triphosphates (NTPs) that are joined together in a synthesized DNA strand using the ligated nucleic acid molecules 110 as templates. The optical signals detected by the optical sensor(s), for instance, are indicative of the sequences of the nucleic acid molecules 110. The sequencer 112 may perform nanopore sequencing. In various cases, the sequencer 112 includes one or more electrical sensors configured to measure an electrical signal (e.g., an electrical current) across a substrate as the ligated nucleic acid molecules 110 are directed through a nanopore extending through the substrate. The electrical signal over time, in various cases, is indicative of the sequences of the nucleic acid molecules 110 in the sample 106. The sequencer 112, in various implementations, is configured to generate the sequence read data 114 as digital data based on the analog signals detected by the sensor(s). For instance, the sequencer 112 includes one or more analog to digital converters (ADCs). In various cases, the sequencer 112 includes at least one processor configured to generate the sequence read data 114.

In some implementations, the sequencer 112 performs RNA sequencing (RNA-seq) on the nucleic acid molecules 110. For example, the nucleic acid molecules 110 include RNA that is extracted from the sample 106. In some examples, the RNA in the nucleic acid molecules 110 is fragmented. In various implementations, complementary DNA (cDNA) is generated using reverse transcriptase, such that the cDNA includes sequences that are complementary to the RNA in the nucleic acid molecules 110 from the sample 106. The cDNA, according to various cases, can be sequenced using the DNA sequencing techniques described above. Accordingly, in some cases, the sequence read data 114 indicates sequences of RNA present in the sample 106, which may be indicative of the transcriptome of the subject 102.

In various cases, the sequencer 112 performs sequencing on a subset of the nucleic acid molecules 110. For instance, the sequencer 112 may perform targeted sequencing on portions of the nucleic acid molecules 110 that correspond to one or more predetermined genes, such as any of the specific genes described herein. Other portions of the genome may be specifically sequenced, such as promoters, hotspots, CpG sites, or other portions of the genome that are not specifically genes but have an impact on genomic expression. The sequencer 112, in some cases, may refrain from sequencing at least a portion of the nucleic acid molecules 110 that do not correspond to the subset.

In various cases, the sequencer 112 performs sequencing on a subset of the cDNA molecules. For instance, the sequencer 112 may perform targeted sequencing on portions of the cDNA molecules that correspond to one or more predetermined genes and/or loci. In some examples, the genes include one or more of CYFRA, CEA, or HE4. In various cases, the genes include one or more of ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, or VEGFB. In some examples, the genes include one or more of TP53, PTEN, POLE, MKI67, FAT3, TAF1, ZFHX3, RPL22, SPTA1, FAM135B, CSMD3, GIGYF2, CSDE1, MLL4, ATR, CTNNB1, USH2A, LIMCH1, RRN3P2, FBXW7, CDH19, USP9X, COL11A1, BCOR, ARID1A, ZNF770, ARID5B, SLC9A11, KRAS, PNN, INPP4A, CTCF, CHD4, AMY2B, RBMX, PPP2R1A, TNFAIP6, PIK3R1, SGK1, HOXA7, METTL14, HPD, MIR1277, CCND1, MECOM, NFE2L2, or ESR1, an estrogen receptor (ER) gene, or a progesterone receptor (PR) gene. Other portions of the genome may be specifically sequenced, such as promoters, hotspots, CpG sites, or other portions of the genome that are not specifically genes but have an impact on genomic expression. The sequencer 112, in some cases, may refrain from sequencing at least a portion of the RNA molecules 110 that do not correspond to the subset.

The data analyzer 116, in some cases, determines a secondary score 118 based on analyzing the sequence read data 114. For instance, the secondary score 118 may be indicative of a likelihood that the subject 102 has the health-related condition. The secondary score 118 may include one or more quantitative values. In various cases, the secondary score 118 includes a multidimensional output. In some examples, the data analyzer 116 compares the secondary score 118 to one or more thresholds. The threshold(s) may be determined based clinical guidelines, clinical data (e.g., data obtained from a population of subjects), data obtained from a database, or the like. In various examples, the threshold(s) are determined based on secondary scores of a population of subjects with a particular health-related condition and/or a population of subjects without the particular health-related condition.

In various cases, the secondary score 118 indicates whether the sequences of the nucleic acid molecules 110 indicated by the sequence read data 114 are associated with the health-related condition (or, optionally, associated with the absence of the health-related condition). For instance, the secondary score 118 may indicate whether the sequence read data 114 is indicative of one or more nucleic acid molecules 110 associated with an increased risk of the health-related condition. In various examples, the secondary score 118 is indicative of one or more mutations (e.g., a presence, a level, etc.) associated with the health-related condition. In various examples, the secondary score 118 is indicative of pathogenic nucleic acids associated with the health-related condition (e.g., an infectious disease). In various cases, the secondary score 118 is indicative of a level of one or more of the nucleic acid molecules 110. For instance, overexpression of a first subset of the nucleic acid molecules 110 and underexpression of a second subset of the nucleic acid molecules 110 may be associated with the health-related condition. The secondary score 118 may be indicative of whether levels of the first subset of the nucleic acid molecules 110 are above the corresponding thresholds and whether levels of the second subset of the nucleic acid molecules 110 are below the corresponding thresholds.

In various examples, the data analyzer 116 may generate the secondary score 118 by inputting the sequence read data 114 into a model. For instance, the data analyzer 116 may include one or more mathematical and/or computer-based models that are configured to determine the secondary score 118 based on the sequence read data 114. In some cases, the data analyzer 116 may include one or more ML models that are pretrained to calculate the secondary score 118. The ML model(s), in some examples, include a logistic regression model, a random forest model, an artificial neural network (ANN), a decision tree, a k-nearest neighbor (KNN) model, a support vector machine (SVM), or a naïve Bayes classifier. For example, parameters of the ML model(s) may have been previously optimized based on training data including features of individuals within a population omitting the subject 102. For instance, the ML model(s) was trained using an unsupervised or semi-supervised learning technique, wherein the parameters were optimized to categorize (e.g., cluster) the features of the population. In some cases, the ML model(s) was trained using a supervised learning technique and pre-classified data, wherein the training data further included ground truth secondary scores of the individuals in the population, such that the parameters were optimized to minimize a loss between predicted secondary scores generated by the ML model(s) based on the features of the population and the ground truth secondary scores experienced by the individuals in the population. To increase training robustness, the population represented by the training data may include individuals without the health-related condition, as well as individuals with a variety of types of presentations of the health-related condition. In some examples, the data analyzer 116 may include a regression model, threshold rule, confidence interval, or other type of statistical model capable of categorizing the secondary score 118 based on the sequence read data 114.

In some examples, the data analyzer 116 determines one or more input features 119. The input feature(s) 119, in various cases, include the preliminary score 117 and/or the secondary score 118. The input feature(s) 119 may include one or more additional features indicative of a likelihood of whether the subject 102 has the health-related condition.

The input feature(s) 119, in some examples, include a copy number state of one or more genetic loci indicated by the sequence read data 114. In various implementations, a number of copies of a predetermined sequence at a given locus in the genome of the subject 102 (also referred to as a “copy number” of the locus) is determined. The copy number state, in various implementations, may indicate copy numbers of one or more loci in the genome of the subject 102. For instance, the copy number state may indicate the presence and/or amount of copies of various sequences present in the genome of the subject 102, which may be due to copy number variation.

In various cases, the input feature(s) 119 are derived based on fragments in the nucleic acid molecules 110, and are therefore referred to as “fragmentomic features.” Examples of fragmentomic features include endpoint positions of the fragments in a reference genome (e.g., right endpoints, left endpoints, etc.), endpoint counts at positions within the reference genome (e.g., right endpoint counts, left endpoint counts, etc.), fragment lengths, end motifs, relative read depths of the fragments, the presence of one or more variants in the fragments, or any combination thereof. Fragmentomic features can be expressed in the spatial domain, in an alternate domain, in a preprocessed form, or any combination thereof.

According to various examples, the sequence read data 114 may represent a genome of the subject 102. Various portions of the sequence read data 114 are aligned with at least one reference sequence (e.g., a reference genome). The aligned data is segmented using at least one segmentation technique (e.g., a circular binary segmentation (CBS) method, a maximum likelihood method, a hidden Markov chain method, a walking Markov method, a Bayesian methods, a long-range correlation method, a change point method, or any combination thereof), thereby generating non-overlapping segments of the sequence read data 114, wherein a sequence associated with a given segment is associated with the same copy number (e.g., a number of instances in which the sequence appears in the segment). Various genetic loci are binned, or otherwise sorted, with respect to the segments of the genome of the subject 102. The copy number state, for instance, is representative of the respective copy numbers associated with the genetic loci. In some cases, the copy number state is dependent on (e.g., assigned based on) a major allele coverage ratio and a minor allele coverage ratio, as well as one or more copy number grid models.

In some implementations, the input feature(s) 119 include the presence or absence of a variant (e.g., a pathogenic variant) in one or more genes associated with classifying the health-related condition. In various cases, the genes include one or more of the genes with potential relevance to a determination of whether the subject 102 has a health-related condition, as listed above. In various implementations, the input feature(s) 119 are indicative of a cellular heterogeneity. For instance, the input feature(s) 119 may be indicative of the heterogeneity associated with a particular cell type (e.g., natural killer (NK) cells), heterogeneity associated with multiple cell types (e.g., all immune cells), or the like.

In some cases, the input feature(s) 119 include additional biomarker data. That is, the input feature(s) 119 may include non-genomic features. For instance, input feature(s) 119 may include data indicating at least one of a histological and/or immunohistological image of the sample 106 or another sample collected from the subject 102, a genomic alteration, or a viral status of the subject 102. The additional biomarker data may be generated based on the sample 106, medical images, or other samples obtained from the subject 102. In some cases, the additional biomarker data includes an image of a stained section of the sample 106 or another sample collected from the subject 102. For instance, the stained section is stained with hematoxylin and eosin (H&E) and/or at least one immunostain. In various examples, the input features(s) 119 includes data indicating the presence of at least one biomarker in another sample collected from the subject 102 (e.g., a blood sample, a urine sample, a saliva sample, etc.). The biomarker may include a nucleic acid, a protein, an enzyme, a metabolite, a hormone, an immunological biomarker, or the like.

In various examples, the input feature(s) 119 include additional clinical data. For instance, the input feature(s) 119 may include data indicating results of at least one of a physical exam, an imaging procedure (e.g., an ultrasound, an MRI, a CT scan, a PET scan, a colonoscopy, an endoscopy, a mammogram, etc.), an electrophysiological test (e.g., an electrocardiogram, an electroencephalogram), a functional test (e.g., to assess the function of an organ or a system), or another assessment performed on the subject 102. In some cases, the input feature(s) 119 include a family history of the pathological condition of the subject 102, lifestyle factors associated with the health-related condition (e.g., smoking history, environmental exposures, etc.), or demographic information of the subject 102 (e.g., age, gender, body mass index (BMI), etc.).

To categorize the health-related condition of the subject 102, a predictive model 120 is configured to generate one or more condition indicator(s) 122 based on the input feature(s) 119. The predictive model 120, for example, may include one or more mathematical and/or computer-based models that are configured to predict the health-related condition indicator based on the input feature(s) 119. For instance, the predictive model 120 may include a regression model, threshold rule, confidence interval, or other type of statistical model capable of categorizing the health-related condition based on the input feature(s) 119. In various cases, the predictive model 120 includes at least one classifier configured to generate the condition indicator(s) 122 based on the input feature(s) 119.

In various implementations, the predictive model 120 includes at least one trained ML model configured to output the condition indicator(s) 122 in response to receiving the input feature(s) 119 in input data. For example, parameters of the ML model(s) may have been previously optimized based on training data including features of individuals within a population omitting the subject 102. For instance, the ML model(s) was trained using an unsupervised or semi-supervised learning technique, wherein the parameters were optimized to categorize (e.g., cluster) the features of the population. In some cases, the ML model(s) was trained using a supervised learning technique and pre-classified data, wherein the training data further included ground truth health-related conditions of the individuals in the population, such that the parameters were optimized to minimize a loss between predicted conditions generated by the ML model(s) based on the features of the population and the ground truth conditions experienced by the individuals in the population. To increase training robustness, the population represented by the training data may include individuals without the health-related condition, as well as individuals with a variety of types of presentations of the health-related condition. Various types of ML models can be included in the predictive model 120, such as a neural network (e.g., a CNN), a nearest-neighbor model, a regression analysis model, a clustering model, a principal component analysis model, a gradient boosting model, a random forest, or any combination thereof. In some cases, the predictive model 120 includes a hybrid model, that includes multiple types of ML models. For instance, the predictive model may include a CNN and a clustering model.

In particular examples, the predictive model 120 includes a clustering model. In various implementations, the clustering model is pre-trained based on training data that includes population features. According to various implementations, the population features include genomic features and/or additional biomarker data of the population. In some cases, the population features further include one or more known conditions and/or prognostic classifications of the population. In various implementations, at least one computing device is configured to cluster the population features. The clustering model, for instance, stores, includes, or otherwise indicates the determined clusters.

In various examples, the population characteristics are defined in a multi-dimensional feature space. In various cases, the feature space has n dimensions (e.g., a dimensionality value of n), wherein n corresponds to the number of feature types included in the population feature. In various cases, data objects representing the population features of the population are plotted or otherwise defined in the feature space. In some examples in which n is greater than two, the data objects are projected onto an m-dimensional feature space using multi-dimensional scaling, wherein m is between 1 and n-1 (inclusive). Multi-dimensional scaling can be achieved using various techniques. For instance, multi-dimensional scaling can be performed using at least one of a statistical method (e.g., t-distributed stochastic neighbor embedding (t-SNE), uniform manifold approximation and projection (UMAP), representation learning (e.g., principal component analysis (PCA), independent component analysis (ICA), etc.), ML-based latent space learning (e.g., autoencoders, transformers, generative adversarial networks, etc.). Accordingly, in some cases, the data objects can be visualized in a Cartesian coordinate system.

Within the feature space (whether it has two or more than two dimensions), the data objects are separated from each other by distances. Various types of distances can be utilized in implementations of the present disclosure. For example, the distances may include Euclidian distances, Manhattan distances, Hamming distances, Minkowski distances, Chebyshev distances, or any combination thereof.

Various clustering techniques can be utilized to generate the clustering model. For instance, the clusters may be generated using k-means clustering, density-based clustering, centroid-based clustering, spectral clustering, distribution-based clustering, hierarchical clustering, or any combination thereof. In some implementations, the clustering model is generated by performing hierarchal clustering on the data objects representing the population features. In various cases, the clusters include two or more data objects that are within proximity of each other (e.g., within a predetermined distance of one another) in the feature space. For instance, a cluster may include two or more data objects that are within a predetermined distance (e.g., Euclidian distance) of one another in the feature space. In some implementations, a data object is included in a cluster if the data object is within an appropriate distance of a linkage criterion representing one or more data objects that are already defined within the cluster. Various implementations of the present disclosure utilize one or more linkage criteria, such as a single-linkage criterion, a complete-linkage criterion, an average-linkage criterion (e.g., a weighted average criterion, an unweighted average criterion), a centroid-linkage criterion, a median linkage criterion, a Ward linkage criterion, a minimum error sum of squares criterion, a min-max criterion, a Hausdorff linkage criterion, a medoid linkage criterion, a minimum energy clustering criterion, or any combination thereof.

In some cases, agglomerative clustering is used to generate the clusters. For example, initially, each data object is defined within the feature space without clustering. Subsequently, pairs of adjacent data objects may be clustered together. In some examples, the process of generating a cluster based on independent data objects in a feature space, or of adding a data object to an existing cluster, may be referred to as “merging.”

In some examples, divisive clustering is used to generate the clusters. For example, the data objects may be defined into a single cluster in the feature space. Subsequently, the single cluster may be divided into multiple clusters. In some instances, the process of dividing a preliminary cluster into multiple subsequent clusters, or of removing a data object from a cluster, may be referred to as “splitting.”

In various cases, each cluster is defined according to a boundary (also referred to as a “border”). In some implementations, data objects outside of the boundary of a cluster are not part of the cluster. Data objects inside of the boundary of the cluster are part of the cluster. Depending on the data objects, the linkage criterion, the feature space, and other characteristics of the training data, the clusters may have irregular shapes within the feature space. In various cases, the clustering model includes the boundaries of the clusters generated based on the data objects defined by the population features.

According to various cases, each cluster in the clustering model is associated with one or more characteristics. The characteristic(s), for instance, are associated with the presence or absence of the health-related condition in the samples associated with the cluster. In some cases, at least one characteristic is defined in at least one dimension of the feature space, such that the clusters are defined according to the health-related condition(s). In some examples, the population features used to define the clusters include characteristics that are beyond the mere categorization of the presence or absence of the health-related condition in the population. Once the clusters are generated based on non-classification features (e.g., genomic features and/or additional biomarker data), characteristics associated with the clusters are subsequently determined. For example, an example cluster may be defined based on the data objects representing the non-condition population features of m members of the population, wherein m is an integer that is greater than one. In various cases, characteristics of the m members of the population are determined. Common characteristics of the population (e.g., the presence or absence of the health-related condition) are determined. For example, if greater than a threshold number of the m members have the health-related condition that is resistant to a predetermined therapy, than resistance to the predetermined therapy may be associated with the example cluster. In various cases, each cluster may be labeled with, or otherwise associated with, one or more characteristics, such as one or more pathological and/or nonpathological conditions. The one or more health-related conditions and/or prognostic features associated with a given cluster form the condition associated with the cluster. In various cases, each cluster in the clustering model is associated with a particular health-related condition.

In various implementations, the health-related condition of the subject 102 is categorized by comparing the input feature(s) 119 of the subject 102 to the clusters in the clustering model. The condition indicator(s) 122 is determined based on a comparison between the input feature(s) 119 and the clusters in the clustering model. In various cases, a data object defined by the input feature(s) 119 of the subject 102 is defined in the feature space of the clustering model. The clustering model, for instance, may determine that the data object is present within the boundary of a particular cluster that was previously defined based on the training data. In some cases, the clustering model determines that the data object is associated with a particular cluster based on a distance between the data object and the particular cluster in the feature space. In some cases, the distance is at least one of a Euclidian distance, a Manhattan distance, a Hamming distance, a Minkowski distance, a Chebyshev distance, or any combination thereof. For instance, the clustering model determines that the distance between the data object and the boundary and/or a centroid of the particular cluster is below a threshold distance. In some examples, the clustering model classifies the health-related condition of the subject 102 into a classification associated with the particular cluster by determining that a distance between at least one data object corresponding to the population features in the cluster is below a threshold distance.

In various cases, the condition indicator(s) 122 of the sample 106 are generated using the input feature(s) 119 and the predictive model 120. In various cases, the predictive model 120 may determine that the subject 102 is associated with one or more conditions and/or prognostic features associated with the cluster in which the input feature(s) 119 belong. In various implementations, the condition indicator(s) 122 indicate a likelihood that the subject 102 has a particular health-related condition. For example, the condition indicator(s) 122 indicate a likelihood that the subject 102 has a cancer type and/or a cancer subtype. In various implementations, the condition indicator(s) 122 are indicative of at least one predicted cell type of the lesion 104 of the subject 102 and/or a predicted tumor progression of the lesion 104. In some cases, the predicted tumor progression may indicate a rate of growth of the lesion 104. In some examples, the predicted tumor progression indicates a rate of growth of one or more predicted cell types of the lesion 104. In various cases, the predicted tumor progression indicates a predicted metastasis profile of the subject 102. The predicted metastasis profile may indicate a time (e.g., a date, a time range) when the lesion 104 will metastasize. In some examples, the condition indicator(s) 122 include a likelihood that the lesion 104 will metastasize (e.g., to the lymph nodes, or to a particular organ) by a given time or an indication that there is greater than a threshold (e.g., 80%) likelihood that the lesion 104 will metastasize by the given time. In various cases, the condition indicator(s) 122 may indicate a prognosis of the subject 102. The prognosis of the subject 102 may indicate a predicted survivability of the subject 102.

In various examples, the condition indicator(s) 122 include a predicted condition (e.g., disease, cancer type, etc. ) of the subject 102; a predicted disease subtype of the subject 102; a predicted survivability of the subject 102; one or more predicted symptoms of the subject 102; a dosage of one or more therapeutic agents (e.g., biologics, chemotherapeutic agents, etc.) predicted to treat the health-related condition of the subject 102, a predicted stage of the predicted disease of the subject 102; a predicted grade of the predicted disease of the subject 102; a predicted activity level of the subject 102 (e.g., a predicted Eastern Cooperative Oncology Group (ECOG) performance status of the subject 102); a predicted body mass index (BMI) of the subject 102; a predicted smoking history of the subject 102; a predicted breast density of the subject 102; a clinical trial that the subject 102 is predicted to qualify (e.g., be eligible) for; or a characteristic of the predicted disease of the subject. For instance, the condition indicator(s) 122 may indicate that the subject 102 is likely to qualify for a clinical trial based on an age, a gender, a disease stage, and previous treatments of the subject 102.

In various examples, the prognostic features may include predicted characteristics of the disease that is experienced by the subject 102, predicted symptoms (e.g., predicted chronic symptoms, such as diabetes, hypertension, etc., or predicted medical events, such as heart attack, stroke, pre-eclampsia, etc.) of the subject 102, predicted causes of the disease, or the like. Accordingly, the health-related condition of the subject 102 can be determined based on the input feature(s) 119.

Various therapies may be specified by the condition indicator(s) 122. Therapies include drug therapy, a targeted therapy, vaccine therapy, stem cell transplantation, blood transfusion, physical therapy, psychiatric therapy, surgery, a behavior modification program, an interventional procedure, a pain management procedure, a minimally invasive procedure, dialysis, or respiratory support. Drug therapy can include one or more anticancer agents (e.g., chemotherapy, radiation therapy, immunotherapy, a targeted therapy, surgery, etc.) In various cases, the condition indicator(s) 122 provide a dosage of one or more therapeutic agents predicted to treat the health-related condition and/or its predicted subtype. Various examples of behavior modification programs include, for instance, a nutritional plan (e.g., a particular diet), a physical exercise plan, Cognitive Behavioral Therapy (CBT), or a stress management plan.

In some implementations, the predictive model 120 is unable to conclusively categorize the health-related condition of the subject 102. For example, the predictive model 120 may determine that the input feature(s) 119 of the subject 102 do not fit within any of the previously defined clusters in the clustering model. In various cases, the predictive model 120 may output an indication that that the categorization of the health-related condition is inconclusive.

A report generator 124 is configured to generate a report 126 based, at least in part, on the condition indicator(s) 122. The report 126, for example, includes consumable data that can inform the care provider(s) 105 about the predicted condition of the subject 102. In various implementations, the report 126 may indicate the results of additional analyses, such as the results of a histological study, whole transcriptome sequencing, cfRNA sequencing, whole exome sequencing, whole genome sequencing, a hotspot panel test, a DNA methylation test, a DNA fragmentation test, an RNA fragmentation test, a microsatellite instability (MSI) test, or a viral status test. The performance of such tests is within the ordinary skill of the art, with additional detail provided elsewhere herein. The report 126, for example, may include a genomic profile of the subject 102 based on various combinations of the above analyses and tests.

In some implementations, the report 126 indicates that a follow-up test of the subject 102 is indicated. For instance, in response to determining that the categorization of the health-related condition of the subject 102 is inconclusive, the report generator 124 may generate the report 126 to indicate that one or more additional tests, such as a physical exam, a histological study, or a nucleic acid sequencing-based test (e.g., genome sequencing, exome sequencing, additional DNA sequencing, RNA sequencing, transcriptome sequencing, etc.) should be performed in order to accurately identify the health-related condition of the subject 102. In some examples, the one or more additional tests may include diagnostic imaging, such as magnetic resonance imaging, computed tomography scan, ultrasound, X-ray, mammogram, positron emission tomography, bone scintigraphy, myelography, virtual colonoscopy, echocardiography, radiography, nuclear medicine, fluoroscopy, or single-photon emission computed tomography. Various examples of follow-up tests appropriate for autoimmune conditions, for instance, include an electroencephalography (EEG), an electrocardiogram (ECG), Holter monitoring, blood pressure monitoring, plethysmography, a urea breath test, respiratory function testing, blood gas analysis, electromyography (EMG), a nerve conduction study (NCS), ultrasound imaging, endoscopy, or a bone density measurement. Examples of follow-up tests appropriate for fetal conditions, for instance, include amniocentesis, fetal electrocardiography, and the like. In some cases, the follow-up test includes performance of another nucleic acid sequencing test.

In various cases, the report 126 is output to a clinical device 128. For example, the report generator 124 transmits the report 126 to the clinical device 128. In various implementations, the clinical device 128 is a computing device that is operated by, owned by, or otherwise associated with the care provider(s) 105. For instance, the clinical device 128 may be a desktop computer, a laptop computer, a smart phone, or some other computing device associated with the care provider(s) 105. The clinical device 128, in various cases, outputs the report 126 to the care provider(s) 105. In some cases, the clinical device 128 includes a display (e.g., a screen) that visually presents the report 126. In various cases, the clinical device 136 includes a speaker that outputs a sound indicative of the report 126. The clinical device 128, in various cases, may output the information in the report 126 using one or more output mechanisms or devices.

The care provider(s) 105 may review the report 126 by interacting with the clinical device 128. The report 126, in various cases, may enhance the clinical decision-making of the care provider(s) 105. For instance, the care provider(s) 105 may prepare and/or administer a therapy to the subject 102 based on the report 126. According to various implementations, the care provider(s) 105 may initiate the therapy and/or refer the subject 102 to another care provider to receive the therapy. In various cases, if the predicted condition of the subject 102 is a disease (e.g., respiratory disease), the care provider(s) 105 may prescribe, recommend, or administer an agent in order to treat the disease the subject 102.

In various implementations, the care provider(s) 105 may develop a diagnosis and/or prognosis of the subject 102 based on the report 126. In various implementations, the care provider(s) 105 may communicate information in the report 126 to the subject 102.

FIG. 1 illustrates various elements that can be embodied in one or more computing devices. For example, at least a portion of the functions of the sequencer 112, the data analyzer 116, the predictive model 120, the report generator 124, or the clinical device 128 are performed by one or more processors in at least one computing device. Examples of computing devices include server computers, desktop computers, laptop computers, tablet computers, mobile phones, wearable devices, Internet of Things (IoT) devices, and the like. In various cases, instructions for performing at least a portion of the functions of these elements are stored in memory and/or in a non-transitory computer readable medium. The instructions, for instance, are executed by the processor(s).

FIG. 1 also illustrates various types of data. For example, the protein data 113, the sequence read data 114, the preliminary score 117, the secondary score 118, the input feature(s) 119, the condition indicator(s) 122, the report 126, or any combination thereof, includes data. The various types of data illustrated in FIG. 1 may be stored, such as in memory or in non-transitory computer readable media. In various implementations, at least a portion of the data is transmitted or otherwise output by one or more computing devices. For example, a computing device may transmit one or more communication signals to another computing device, wherein the communication signal(s) encode at least a portion of the data. Examples of communication signals include electromagnetic signals, optical signals, ultrasonic signals, optical signals, and electrical signals. For example, communication signals can be transmitted wirelessly and/or in a wired fashion. The communication signals, for instance, are transmitted over one or more wireless channels and/or one or more wired channels (e.g., optical cabling, electrical cabling, etc.). In various cases, the communication signal(s) are transmitted over one or more communication networks. A communication network, for instance, may be defined according to one or more physical channels, such as one or more frequency spectra. In some cases, a communication network is defined according to one or more communication protocols and/or standards. Examples of communication networks include fiber optic networks, Institute of Electrical and Electronics Engineers (IEEE) networks (e.g., WI-FI™ networks, WiMAX networks, BLUETOOTH™ networks, etc.), cellular networks (e.g., a 3^{rd} Generation Partnership Project (3GPP) radio network, such as a Long Term Evolution (LTE) network, a New Radio (NR) network; or a cellular core network such as a 3^{rd} Generation (3G) core, a 4^{th} Generation (4G) core, a 5^{th} Generation (5G) core, etc.), ultrasonic networks, and the like. In some cases, the data is broadcasted from one device to multiple other devices. In some cases, the data is unicasted from one device to another device. For instance, various forms of data described herein may be transmitted via a peer-to-peer (P2P) connection.

A particular example will now be described with reference to FIG. 1. In this example, the subject 102 presents to a clinical environment due to unexplained fatigue and pain. The care provider(s) 105 may, without ordering imaging of the subject 102, obtain the sample 106 from the blood of the subject 102. The protein detector 111 may generate protein data 113 based on proteins 108 in the blood sample 106 of the subject 102. For example, the protein data 113 may represent the presence and/or the level of one or more proteins associated with lung cancer. Examples of proteins associated with lung cancer include CYFRA, CEA, and HE4. In various examples, the data analyzer 116 may determine a preliminary score 117 indicative of a likelihood, based on the protein data 113, that the subject 102 has lung cancer.

In some examples, the data analyzer 116 determines, based on the preliminary score 117, that the subject 102 has a low likelihood of having lung cancer. For instance, the data analyzer 116 may determine input feature(s) 119 based on identifying one or more of: attributes of the protein data 113, the preliminary score 117, or other features of the subject 102 and/or the sample 106 that are indicative of lung cancer. The predictive model 120 may generate the condition indicator(s) 122 based on the input feature(s) 119. The condition indicator(s) 122 may indicate that the subject 102 is unlikely to have lung cancer. Accordingly, the report generator 124 may output a report 126 indicative of the condition indicator(s) 122 to a clinical device 128. Based on receiving the report 126 using the clinical device 128, the care provider(s) 105 may advise the subject 102 to not undergo additional testing procedures.

In some examples, the data analyzer 116 determines, based on the preliminary score 117, that the likelihood of the subject 102 having lung cancer is above a particular threshold. Accordingly, the data analyzer 116 may output an indication to identify the sequence read data 114 of the sample 106. The sequencer 112, in various cases, identifies sequences of the nucleic acid molecules 110 in the sample 106. The data analyzer 116 may determine a secondary score 118 based on the sequence read data 114. The secondary score 118 is indicative of one or more DNA and/or RNA molecules associated with lung cancer. The data analyzer 116 may determine input feature(s) 119 based on identifying one or more of: attributes of the protein data 113 and/or the sequence read data 114, the preliminary score 117, the secondary score 118, or other features of the subject 102 and/or the sample 106 that are indicative of lung cancer. The predictive model 120 may generate the condition indicator(s) 122 based on the input feature(s) 119. The condition indicator(s) 122 may indicate that the likelihood of the subject 102 having lung cancer is above a threshold. In some examples, the condition indicator(s) 122 indicates one or more follow-up tests to identify a location and/or a characteristic (e.g., a subtype) of the lung cancer. Accordingly, the report generator 124 may output a report 126 indicative of the condition indicator(s) 122 to a clinical device 128. Based on receiving the report 126 using the clinical device 128, the care provider(s) 105 may perform one or more diagnostic imaging procedures (e.g., a CT scan) to identify a location of the lesion 104.

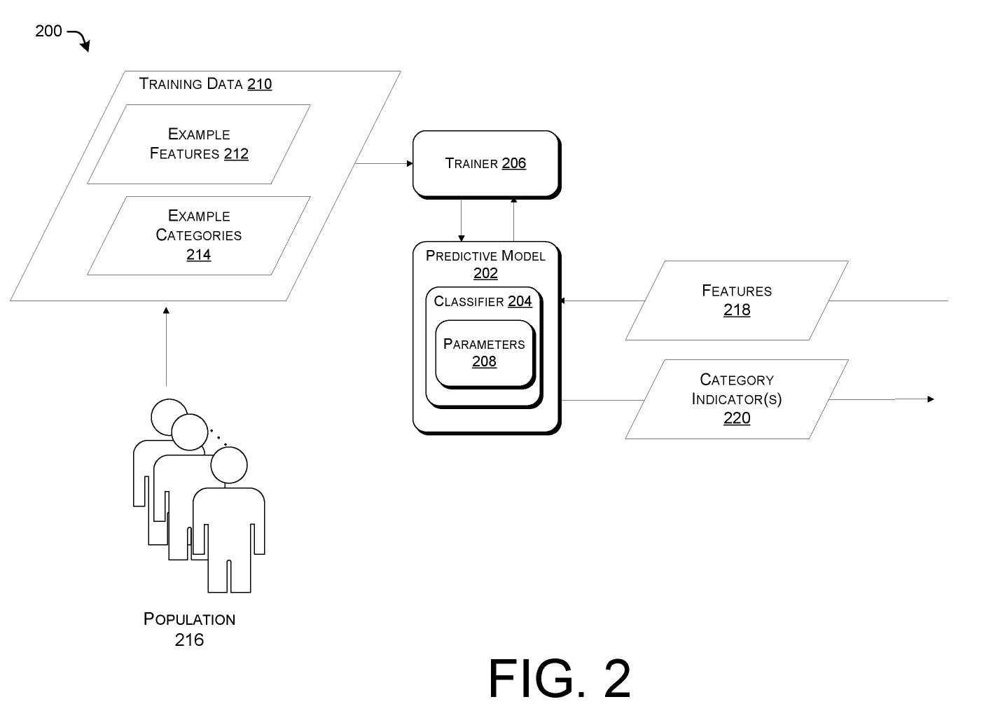

FIG. 2 illustrates an example environment 200 for training and utilizing a predictive model 202 to determine a health-related condition of a subject based on a non-sequencing-based analysis, and optionally based on a sequencing based analysis. The predictive model 202, for instance, is the predictive model 120 described above with reference to FIG. 1. In various implementations, the predictive model 202 includes a classifier 204, which may include one or more ML models. A trainer 206, for instance, is configured to optimize various parameters 208 of the classifier 204 based on training data 210.

The training data 210 includes example features 212 and example categories 214. The example features 212, in various cases, are obtained based on protein molecules and/or nucleic acid molecules of individuals within a population 216. In some examples, the example features 212 may include preliminary scores (e.g., the preliminary score 117) and/or secondary scores (e.g., the secondary score 118) of individuals within the population 216. The example categories 214 may include categorizations of pathologies experienced by the individuals within the population 216. For example, the example categories 214 may be generated based on clinical evaluations of the individuals within the population 216, such as by one or more care providers.

The classifier 204 include one or more model types. For instance, the classifier 204 include an artificial neural network. An artificial neural network includes various layers that respectively process input data. For example, an artificial neural network includes an input layer, one or more hidden layers, and an output layer. The input layer performs a pre-processing operation on the input data. The hidden layer(s) may perform various processing operations on the output from the input layer. The output layer, in various cases, processes the output from the hidden layer(s). Each layer, in some cases, includes one or more nodes, which are defined by individual operations. In various cases, the hidden layer(s) include nodes that are connected to each other in parallel and/or series. Examples of artificial neural networks include feedforward neural networks, multi-layer perceptrons (MLPs), convolutional neural networks (CNNs), and backpropagation models. In various implementations, the operations performed by the layers and/or nodes within an artificial neural network included in the classifier 204 is defined according to the parameters 208. For example, the parameters 208 may include weights, thresholds, filters, kernels, or other data objects that are utilized to perform operations of the classifier 204.

In some implementations, the classifier 204 include a nearest-neighbor model. One example of a nearest-neighbor model includes a k-nearest neighbor model. For example, a nearest-neighbor model defines various “neighbors,” which are points within a feature space, with associated class labels. When a new data point is mapped to the feature space, the new data point is classified based on the proximity (e.g., Euclidian distance, Manhattan distance, Minkowski distance, etc.) of its “neighbors” to the new data point as well as their associated classes. In some cases, the new data point is classified as belonging to a particular class if greater than a threshold number of neighbors within a threshold distance of the new data point are members of the class. For instance, the parameters 208 may include k (e.g., the number of neighbors compared to the new data point), the threshold distance, and so on.

In various cases, the classifier 204 include a regression analysis model. The regression analysis model, for example, is defined by a regression function that defines relationships between one or more independent variables and one or more dependent variables. The regression function may further define one or more unknown parameters that define a relationship between the independent and dependent variables. In various implementations, the unknown parameters and/or the type of regression function (e.g., linear, quadratic, etc.), is defined according to the parameters 208.

In some cases, the classifier 204 include a clustering model. In various cases, a clustering model maps various data points (e.g., training data) to a feature space. Based on the proximity of groups of those data points in the features pace, one or more “clusters” are defined. An additional data point may be classified according to one or more of the clusters based on its proximity to the clusters (e.g., a center of the clusters, a boundary of the cluster, etc.). Examples of clustering models include k-means clustering, mean-shift clustering, expectation-maximization (EM) clustering, and agglomerative hierarchical clustering. The parameter(s) 208, for example, include a threshold proximity within which a new data point is classified within a cluster, a density of points used to define a cluster, and the like.

In various examples, the classifier 204 include a principal component analysis model. In various implementations, a principal component analysis defines a collection principal components of unit vectors within a coordinate space based on a data set (e.g., training data). The model, for example, is an orthogonal linear transformation of the data set. Various weights of the model, for example, are included in the parameter(s) 208.

The classifier 204, in some implementations, includes a gradient boosting model. For example, the gradient boosting model is defined as a collection of prediction models (e.g., decision trees) that iteratively classify observed data. In various cases, the type of prediction model, weights in the prediction models, and the like, are defined by the parameter(s) 208.

The classifier 204, for example, includes a random forest. The random forest, for instance, includes multiple decision trees that classify data in an ensemble fashion. In various implementations, the decision trees are defined by the parameter(s) 208.

In various implementations of the present disclosure, the trainer 206 is configured to optimize the parameters 208 based on the training data 210. For example, the trainer 206 may input first example features (corresponding to a first individual among the population 216) among the example features 212 into the predictive model 202, and may receive a predicted category. The trainer 206 may compute a loss (e.g., determine a discrepancy) between a first example category (corresponding to the first individual) among the example categories 214 and the predicted category. Further, the trainer 206 may alter the parameters 208 in order to minimize the loss. In various cases, the trainer 206 optimizes the parameters 208 iteratively based on the entire set of the training data 210.

In various implementations, the optimization of the parameters 208 enables the predictive model 202 to identify predictive attributes of the example features 212 that are correlated to or otherwise associated with the example categories 214. For instance, the predictive model 202 may determine that a particular amino acid sequence represented in the example features 212 is highly correlated with adenosarcoma. The predictive model 202 may therefore classify cancers based on features outside of the example features 212 by recognizing or otherwise identifying the predictive attributes.

Once the parameters 208 are optimized, the predictive model 202 may be ready to classify a new set of data. For example, the predictive model 202 may receive input data including features 218 of a subject. The features 218, for instance, may include one or more of the predictive attributes. The predictive model 202 may perform various operations on the input data based on the trained classifier 204 and the optimized parameters 208. In various cases, the predictive model 202 outputs output data including one or more category indicators 220 based on the features 218. The category indicator(s) 220, for instance, include one or more predicted categories of a cancer experienced by the subject.

Although FIG. 2 is primarily described as referring to supervised learning, implementations are not so limited. In various cases, the training data 210 omits the example categories 214 and the trainer 206 is configured to optimize the parameters 208 using the example features 212 and an unsupervised learning technique.

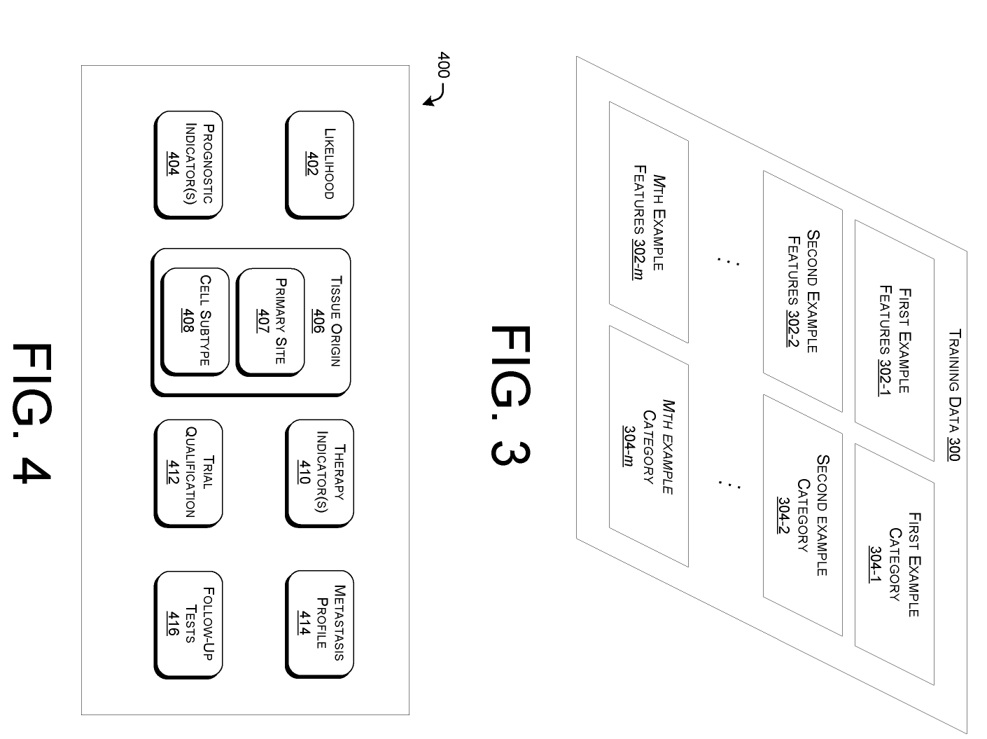

FIG. 3 illustrates an example of training data 300 utilized to train one or more ML models. For example, the training data 300 may be the training data 210 described above with reference to FIG. 2.

The training data 300, in various cases, may represent m samples, wherein m is a positive integer. In some cases, the m samples are respectively obtained from m individuals within a population, although implementations are not so limited. For example, in some cases, multiple samples may be obtained from the same individual at different times.

The training data 300 includes first to mth example features 302-1 to 302-m. For example, the first to mth example features 302-1 to 302-m include features derived from proteins and/or nucleic acid molecules in the respective m samples.

The training data 300 may further include first to mth example categories 304-1 to 304-m. The first to mth example categories 304-1 to 304-m, for instance, include categories or classifications of cancers represented by the m samples.

FIG. 4 illustrates an example report 400 summarizing predicted categories of a cancer of a subject. In various cases, the report 400 is the report 126 described above with reference to FIG. 1. The report 400, for instance, may be displayed to a patient and/or care provider. In some cases, the report 400 is generated based on features of a sample (e.g., a liquid biopsy sample) obtained from the subject.

The report 400 includes a likelihood 402 of the cancer. The likelihood 402, for instance, indicates a likelihood that the subject 102 has a particular cancer type and/or subtype.

In some examples, the report 400 includes one or more prognostic indicators 404. The prognostic indicator(s) 404, for instance, indicate a prognosis of the subject in view of the categorized cancer. For example, the prognostic indicator(s) 404 may indicate a survivability, a recoverability, a quality of life indicator, or other information indicative of the prognosis of the subject.

The report 400 includes a tissue origin 406 of the cancer. The tissue origin 406, for instance, indicates a primary site 407, cell subtype 408, or any combination, of the cancer.

In various cases, the report 400 includes one or more therapy indicators 410. For instance, the therapy indicator(s) 410 convey whether the cancer is predicted to be resistant to one or more predetermined therapies and/or whether the cancer is predicted to be responsive to one or more predetermined therapies.

The report 400 may include a trial qualification 412 of the subject. The trial qualification 412, for instance, indicates whether the subject is predicted to qualify for a predetermined clinical trial.

The report 400, in various implementations, includes a metastasis profile 414 of the subject. The metastasis profile 414, for instance, indicates a likelihood that the cancer will metastasize (e.g., at a particular point in time), one or more tissues in which the cancer is predicted to metastasize, or the like.

In various cases, the report 400 includes recommended follow-up tests 416. For example, the report 400 may include a recommendation to perform whole genome sequencing on the subject, particularly in cases if the cancer cannot be categorized above a threshold certainty.

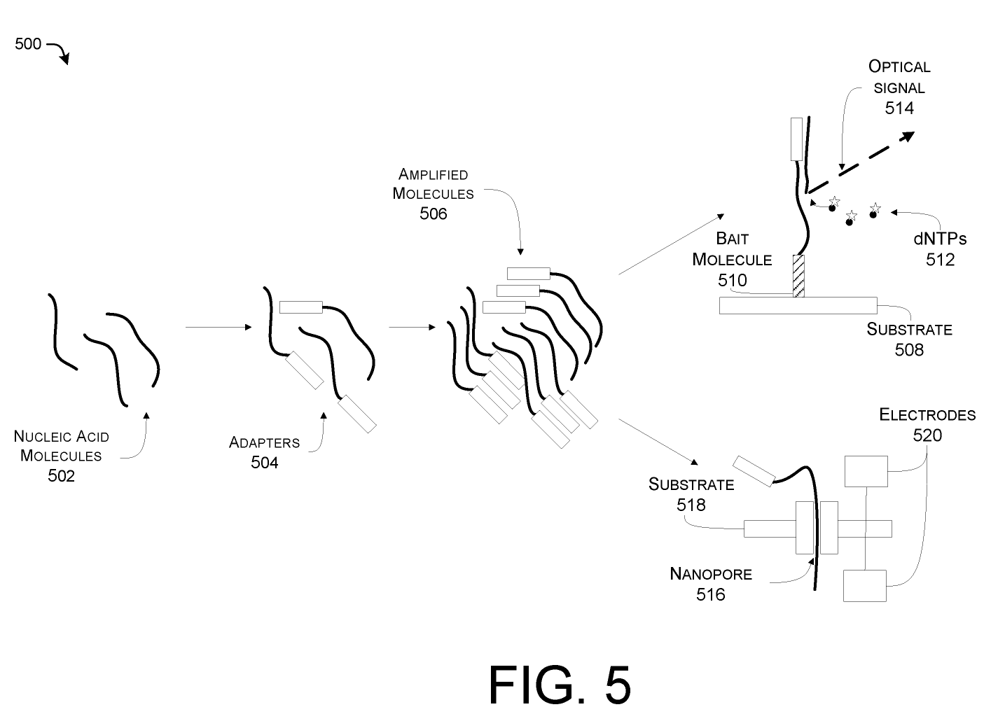

FIG. 5 illustrates an example environment 500 for sequencing various nucleic acid molecules 502. In various implementations, the nucleic acid molecules 502 include cfDNA and/or gDNA. For instance, the nucleic acid molecules 502 may include ctDNA. The nucleic acid molecules 502, in various cases, are extracted from a sample, such as a biological sample obtained from a subject. In some implementations, the nucleic acid molecules 502 include DNA that is complementary to RNA present in the sample.

The nucleic acid molecules 502, in various cases, are ligated with adapters 504. For examples, the adapters 504 are hybridized to the nucleic acid molecules 502. The adapters 504, for example, include additional nucleic acid molecules. In various implementations, the adapters 504 have a shorter length than the nucleic acid molecules 502 being sequenced. For instance, the adapters 504 include amplification primers, flow cell adapter sequences, substrate adapter sequences, or sample index sequences. Although FIG. 5 illustrates adapters 504 being ligated to one end of each of the nucleic acid molecules 502, implementations are not so limited. For example, the adapters 504 may be ligated to both ends of each of the nucleic acid molecules 502.

In various examples, the nucleic acid molecules 502 ligated with the adapters 504 are amplified in order to generate amplified molecules 506. Various amplification techniques can be performed. For instance, the amplified molecules 506 are generated using PCR, a non-PCR amplification technique, an isothermal amplification technique, or any combination thereof.

Amplified molecules 506 may be captured by bait molecules 510 and sequenced. In some implementations, the amplified molecules 506 are sequenced via sequencing-by-synthesis. In various cases, fluorescently tagged deoxyribonucleotide triphosphates (dNTP) 512 are utilized to synthesize a strand that is complementary to DNA strands bound to the substrate 508. When a dNTP 512 is added to the strand (e.g., by an enzyme), the dNTP 512 emits an optical signal 514. In various implementations, the frequency of the optical signal 514 is dependent on the type of dNTP 512 from which the optical signal 514 is emitted. By detecting the optical signals 514 as the strand is being synthesized, the sequence of the original nucleic acid molecules 502 can be derived.

In some implementations, the amplified molecules 506 are sequenced via nanopore sequencing. For instance, the amplified molecules 506 are directed through a nanopore 516 extending through a substrate 518. In various cases, the amplified molecules 506 are negatively charged, such that they can be directed through the nanopore 516 by imposing an electrical field across the substrate 518. In various cases, the amplified molecules 506 and the nanopore 516 are in the presence of a charged solution. Thus, charged solutes traveling through the nanopore 516 can be monitored by reviewing an electrical signal (e.g., a current) sensed between electrodes 520 on either side of the substrate 518. As an amplified molecule 506 is directed through the nanopore 516, the individual bases within the amplified molecule 506 will block the nanopore 516, which may decrease the amount of charged solutes traveling through the nanopore 516 and consequently, the magnitude of the electrical signal detected by the electrodes 520. Each of the four types of bases within the amplified molecules 506, may block the nanopore 516 to a different extent. Therefore, the sequence of the nucleic acid molecules 502 can be derived by analyzing the measured electrical signal with respect to time as the amplified molecules 506 are directed through the nanopore 516.

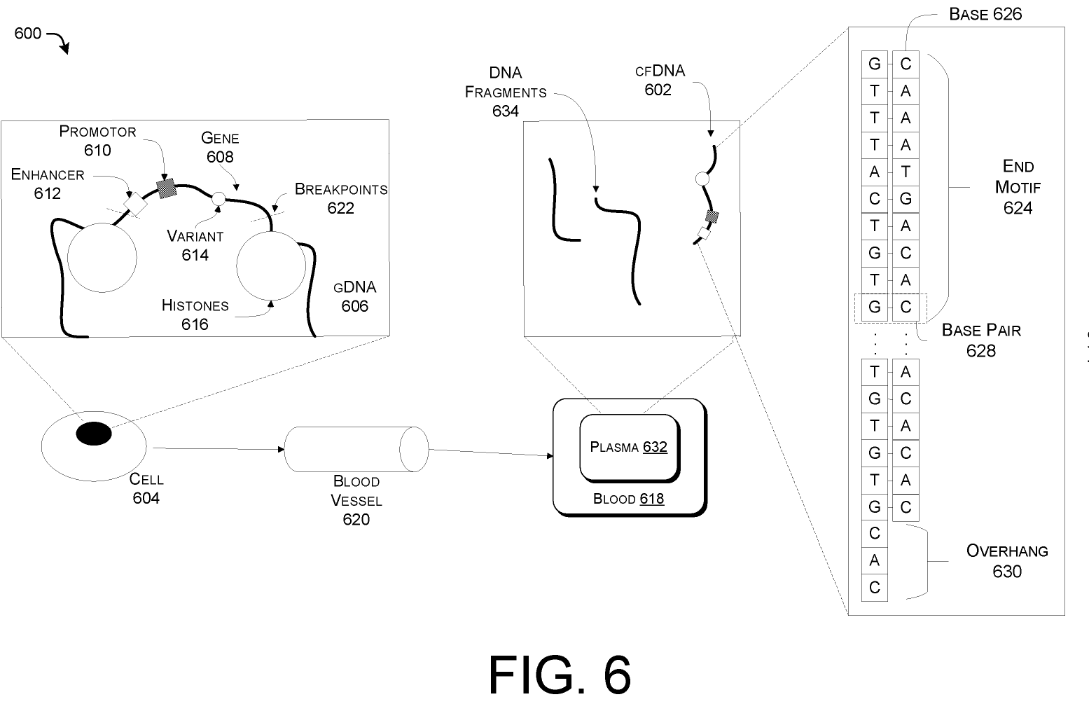

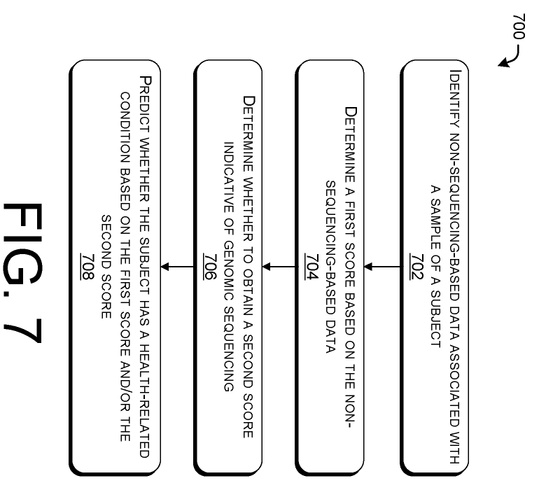

FIG. 6 illustrates an example environment 600 illustrating cfDNA 602, which can be utilized to identify a health-related condition of a subject. For instance, the cfDNA 602 may be included in the nucleic acid molecules 110 described above with reference to FIG. 1.

In various implementations, a cell 604 within the subject includes genomic DNA (gDNA) that is expressed by the cell 604. In various implementations, the cell 604 is associated with the health-related condition. In some cases, the cell 604 is an immune cell. In some cases, the cell 604 is a cancer cell. In some cases, the cell 604 is a normal (e.g., a healthy cell, a non-cancer cell, etc.) that is affected by the health-related condition. For instance, the cell 604 may be damaged during metastasis of the lesion 104 of the subject 102. For example, the gDNA 606 may include various sequences, such as a gene 608, a promoter 610, an enhancer 612, and a variant 614. For example, the variant 614 is part of the gene 608. In addition, various epigenetic factors impact expression of the gene 608 as well as other genes within the gDNA 606. For example, the gDNA 606 may be packaged within the nucleus of the cell 604 with various histones 616. When the gene 608 is expressed, a portion of the gDNA 606 including the gene 608, the promotor 610, the enhancer 612, and the variant 614 may be exposed to proteins within the nucleus, such as RNA transcriptase. In various cases, the portion of the gDNA 606 is unwrapped or otherwise unpackaged from the histones 616. Thus, the expression of the gene 608 (e.g., the amount of mRNA generated by RNA transcriptase based on the gene 608 within the cell 604) is linked to the frequency or time at which the portion of the gDNA 606 is exposed.

The cell 604, for example, may die. The contents of the cell 604, including the gDNA 606, may be released. In various cases, the gDNA 606 is released into blood 618 that flows through a blood vessel 620 of the subject. When the gDNA 606 is released from the nucleus of the cell 604, the gDNA 606 is degraded due to various biophysical and/or biochemical factors. For example, the blood 618 may include various enzymes that cut the gDNA 606 into the cfDNA 602. In various cases, other mechanical, chemical, or thermal conditions in the blood 618 divide the gDNA 606 into the cfDNA 602. For example, these conditions divide the gDNA 606 into fragments at various breakpoints 622.

Notably, the presence and location of the histones 616 may impact the sequences of the cfDNA 602 that are observed in the blood 618. The breakpoints 622, for example, are more likely to occur at edges of a sequence of the gDNA 606 that is exposed by the histones 616. Therefore, the sequence of the cfDNA 602 is indicative of the expression of mRNA and other functional RNA in the cell 604. By reviewing the cfDNA 602, the expression of the cell 604 can be determined without performing RNA sequencing, in some cases. In various examples, the expression of the cell 604 is relevant to the health-related condition of the subject.

In addition, the sequences at or near the breakpoints 622 are indicative of expression of the cell 604. For example, the cfDNA 602 may include an end motif 624. The end motif 624 may be defined as a sequence of bases 626 and/or base pairs 628 that extend from an end of the cfDNA 602. The end motif 624, for example, has a predetermined length that is in a range of 1 to 30 bases and/or base pairs. In various implementations, the cfDNA 602 is a double-stranded DNA molecule with an overhang 630. The overhang 630, for instance, includes one or more bases 626 of one ssDNA molecule that extends beyond the corresponding end of the other ssDNA molecule. In some cases, the end motif 624 is defined as the sequence of bases in a single ssDNA within the cfDNA 602 or a sequence of complementary base pairs in both ssDNA within the cfDNA 602. As described herein, the term “endpoint” may refer to at least one of the bases 626 in the end motif 624 and/or overhang 630 of a DNA fragment in a sample.

In various implementations, the cfDNA 602 is obtained from a sample of plasma 632 in the blood 618 of the subject. The plasma 632, for example, includes various DNA fragments 634 including the cfDNA 602. In some cases, the DNA fragments 634 include various types of cfDNA, such as microbial cell-free DNA (cfmDNA) and/or cfDNA released from fetal cells.

By sequencing the cfDNA 602, various fragmentomic features may be obtained. These fragmentomic features can be utilized to categorize the cell 604, thereby identifying a health-related condition of the subject from which the cell 604 was present. In various cases, the fragmentomic features include the presence of at least a portion of the gene 608 in the cfDNA 602. In some cases, the fragmentomic features include the presence of at least a portion of the promotor 610, the enhancer 612, or the variant 614 in the cfDNA 602. In some cases, the fragmentomic features include the presence or sequence of the end motif 624. Other fragmentomic features are described elsewhere herein.

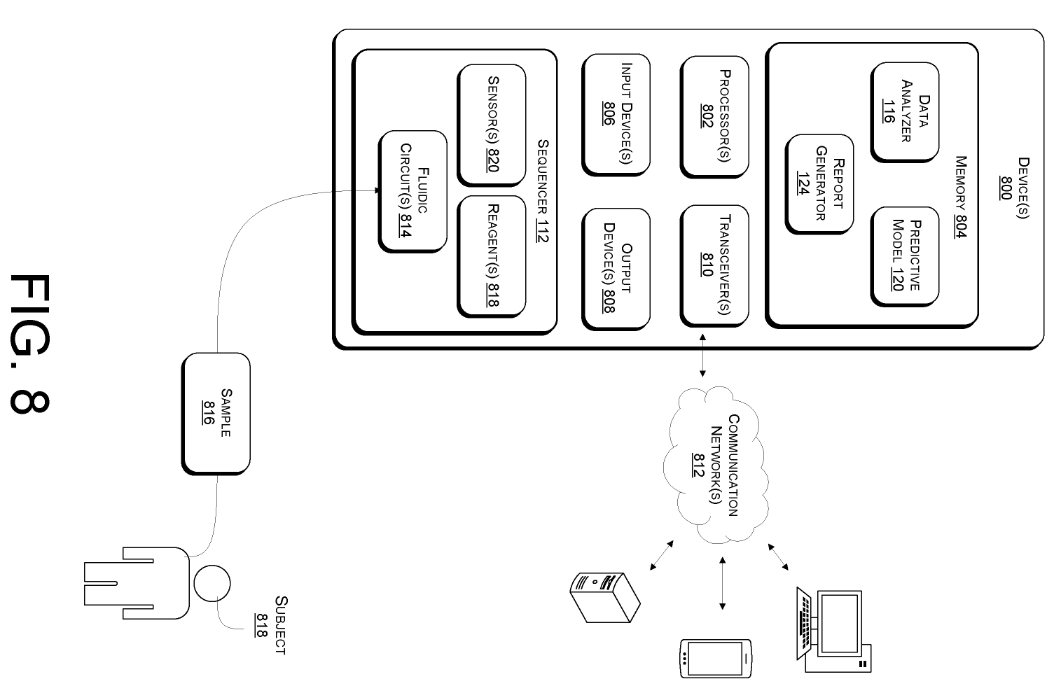

FIG. 7 illustrates an example process 700 for identifying a health-related condition of a subject based on a non- genomic sequencing-based analysis, and optionally based on a genomic sequencing-based analysis. In various implementations, the process 700 is performed by an entity including at least one processor, at least one computing device, a medical device, the sequencer 112, the analyzer 116, the predictive model 120, the report generator 124, the clinical device 128, or any combination thereof.

In some examples, at 702, the entity identifies non-genomic sequencing-based data indicative of proteins in a sample of a subject. For instance, the entity receives a plurality of proteins in a first portion of the sample of the subject. The sample may include a liquid sample (e.g., a blood sample, a urine sample, a saliva sample, etc.). The proteins, for instance, include peptides and/or protein fragments. A capture agent (e.g., an antibody, an antibody fragment, an aptamer, a protein, a peptide, an enzyme, a tag, etc.) may bind the proteins. The capture agent, in some examples, is linked to a detectable label. The non-sequencing-based data may be indicative of the presence and/or a level of a subset of the proteins in the first portion of the sample.

In some examples, at 702, the entity identifies non-genomic sequencing-based data indicative of nucleic acid molecules in a sample of the subject. For instance, the entity receives a plurality of nucleic acid molecules in the first portion of the sample of the subject. The nucleic acid molecules, for instance, include DNA (e.g., genomic DNA) and/or RNA from the sample. In some examples, the nucleic acid molecules include cfDNA and/or ctDNA. In some instances, the entity determines a DNA methylation profile of the nucleic acid molecules in the first portion of the sample of the subject. The DNA methylation profile may indicate a presence, a level, or a pattern of DNA methylation at one or more loci in the genome of the subject.

At 704, the entity determines a first score based on the non-genomic sequencing-based data. In some examples, the first score is indicative of a presence and/or a level of one or more proteins associated with the health-related condition. In some examples, the first score in indicative of a non-genomic sequencing-based analysis of one or more nucleic acid molecules associated with the health-related condition. In various examples, the entity determines the first score by inputting the non-genomic sequencing-based data into a model (e.g., a ML model). Accordingly, the first score may be indicative of a likelihood of the subject having the health-related condition.

At 706, the entity determines whether to identify genomic sequencing data of the sample of the subject based on the first score. For instance, the entity may compare the first score to one or more thresholds. In some examples, the entity may determine that, based on the first score, the subject is unlikely to have the health-related condition. Accordingly, the entity may determine not to obtain the second score of the subject. In some examples, the entity may not be able to determine, based on the first score, whether the subject has or does not have the health-related condition. Accordingly, the entity may determine that genomic sequencing may provide pertinent information about the health-related condition of the subject. In various examples, the entity may compare the first score to one or more thresholds to determine whether to obtain a second score indicative of the genomic sequencing data. The second score, in various implementations, is based on sequence read data indicative of nucleic acids in a second portion of the sample of the subject.

In various implementations in which the entity determines to obtain the second score of the subject, the entity receives a plurality of nucleic acid molecules in a second portion of the sample of the subject. The nucleic acid molecules, for instance, include DNA (e.g., genomic DNA) and/or RNA from the sample. In some examples, the nucleic acid molecules include cfDNA and/or ctDNA. One or more adapters are ligated onto at least some of the nucleic acid molecules. The ligated molecules are amplified and captured. In various cases, all or a subset of the captured molecules are sequenced to obtain a plurality of sequence reads that represent the sequenced amplified nucleic acid molecules, thereby generating the sequence read data. The entity may analyze (e.g., determine a level of) one or more nucleic acids associated with the health-related condition. The second score, in some cases, is indicative of the one or more nucleic acids.

At 708, the entity predicts whether the subject has a health-related condition based on the first score and/or the secpmd score. In various cases, the entity inputs the first score and/or the second score into a machine learning (ML)-based classifier to predict whether the subject has the health-related condition. The ML-based classifier, for instance, is pre-trained based on data obtained from a population of individuals that omits the subject. In some cases, the classifier includes at least one of an artificial neural network (ANN), a logistic regression model, a decision tree, a k-nearest neighbor (KNN) model, a support vector machine (SVM), or a naïve Bayes classifier. In some cases, the classifier outputs a likelihood that the subject has a particular health-related condition (or the absence of a particular health-related condition). In some cases, the classifier outputs an indication that the subject has the particular health-related condition (or its absence) when the likelihood exceeds a threshold likelihood.

FIG. 8 illustrates one or more devices 800 configured to perform various operations described herein. The device(s) 800 include one or more processor(s) 802. In some implementations, the processor(s) 802 includes a central processing unit (CPU), a graphics processing unit (GPU), both CPU and GPU, or other processing unit or component known in the art.

The processor(s) 802 is operably connected to memory 804. In various implementations, the memory 804 is volatile (such as random access memory (RAM)), non-volatile (such as read only memory (ROM), flash memory, etc.) or some combination of the two. The memory 804 stores instructions that, when executed by the processor(s) 802, causes the processor(s) 802 to perform various operations. In various examples, the memory 804 stores methods, threads, processes, applications, objects, modules, any other sort of executable instruction, or a combination thereof. In some cases, the memory 804 stores files, databases, or a combination thereof. In some examples, the memory 804 includes, but is not limited to, RAM, ROM, electrically erasable programmable read-only memory (EEPROM), flash memory, or any other memory technology. In some examples, the memory 804 includes one or more of CD-ROMs, digital versatile discs (DVDs), content-addressable memory (CAM), or other optical storage, magnetic cassettes, magnetic tape, magnetic disk storage or other magnetic storage devices, or any other medium which can be used to store the desired information and which can be accessed by the processor(s) 802. For instance, the memory 804 stores instructions that, when executed by the processor(s) 802, causes the processor(s) 802 to perform operations of the analyzer 116, the predictive model 120, and the report generator 124.

The processor(s) 802 is operably connected to one or more input devices 806 and one or more output devices 808. Collectively, the input device(s) 806 and the output device(s) 808 function as an interface between at least one user and the device(s) 800. The input device(s) 806 is configured to receive an input from a user and includes at least one of a keypad, a cursor control, a touch-sensitive display, a voice input device (e.g., a microphone), a haptic feedback device (e.g., a gyroscope), or any combination thereof. The output device(s) 808 includes at least one of a display, a speaker, a haptic output device, a printer, or any combination thereof. In various examples, the processor(s) 802 causes a display among the input device(s) 806 to visually output various data described herein. In some implementations, the input device(s) 806 includes one or more touch sensors, the output device(s) 808 includes a display screen, and the touch sensor(s) are integrated with the display screen.

In various implementations, the processor(s) 802 is operably connected to one or more transceivers 810 that transmit and/or receive data over one or more communication networks 812. For example, the transceiver(s) 810 includes a network interface card (NIC), a network adapter, a local area network (LAN) adapter, or a physical, virtual, or logical address to connect to the various external devices and/or systems. In various examples, the transceiver(s) 810 includes any sort of wireless transceivers capable of engaging in wireless communication (e.g., radio frequency (RF) communication). For example, the communication network(s) 812 includes one or more wireless networks that include a 3rd Generation Partnership Project (3GPP) network, such as a Long Term Evolution (LTE) radio access network (RAN) (e.g., over one or more LTE bands), a New Radio (NR) RAN (e.g., over one or more NR bands), or a combination thereof. In some cases, the transceiver(s) 810 includes other wireless modems, such as a modem for engaging in WI-FI®, WIGIG®, WIMAX®, BLUETOOTH®, or infrared communication over the communication network(s) 812.

The device(s) 800 may further include the sequencer 112. In various implementations, the sequencer 112 includes one or more fluidic circuits 814 configured to receive a sample 816 derived from a subject 818. The sequencer 112, in various cases, may be configured to generate data indicative of one or more sequences of nucleic acid molecules (e.g., DNA and/or RNA) present in the sample 816. In various cases, the sequencer 112 introduces one or more reagents 819 to the fluidic circuit(s) 814 in order to prepare for and perform sequencing of the nucleic acid molecules. Further, the sequencer 112 may include one or more sensors 820 disposed on the fluidic circuit(s) 814 and configured to measure or otherwise detect detection signals from the fluidic circuit(s) 814, which may be indicative of the sequences of the nucleic acid molecules. According to various implementations, the sensor(s) 820 may further include one or more ADCs. The sequencer 112, in various cases, outputs sequence read data to the processor(s) 802 for additional processing.

### Example Clauses

1. A method, including: dividing a liquid biopsy sample obtained from a subject into a first portion and a second portion; providing a plurality of proteins obtained from the first portion of the liquid biopsy sample; detecting the plurality of proteins, thereby generating protein data; generating a first score based on the protein data; determining that the first score is greater than a threshold; and based on determining that the first score is greater than the threshold: providing a plurality of nucleic acid molecules obtained from the second portion of the liquid biopsy sample; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, all or a subset of the captured amplified nucleic acid molecules to obtain a plurality of sequence reads that represent the sequenced amplified nucleic acid molecules, thereby generating sequence read data; receiving, at one or more processors, the sequence read data for the plurality of sequence reads; generating, by the one or more processors, input features based on at least one of the protein data and the sequence read data; and determining, using a classifier executed by the one or more processors, a likelihood that the subject has at least one type of cancer based on the input features.
2. The method of clause 1, wherein the plurality of proteins include at least one of CYFRA, CEA, or HE4.
3. The method of clause 1 or 2, wherein the subject lacks any apparent disease or other pathological condition.
4. The method of any of clauses 1-3, wherein the protein data includes measurements of the plurality of proteins.
5. The method of any of clauses 1-4, wherein the sequence read data indicates a DNA methylation profile of the plurality of nucleic acid molecules.
6. The method of any of clauses 1-5, wherein the plurality of nucleic acid molecules include DNA fragments obtained from the second portion of the liquid biopsy sample, and wherein the sequence read data is indicative of at least one of: endpoint counts of the DNA fragments, endpoint positions of the DNA fragments, lengths of the DNA fragments, read depth of the DNA fragments, or sequences of the DNA fragments.
7. The method of any of clauses 1-6, wherein generating the first score includes: inputting the protein data into a logistic regression model configured to generate a probability that the protein data is indicative of the at least one type of cancer based on the protein data; and predicting the first score of a plurality of scores associated with the liquid biopsy sample based on the probability generated by the logistic regression model.
8. The method of any of clauses 1-7, wherein the input features are generated based on the protein data and the sequence read data.
9. A method, including: identifying protein data of a sample obtained from a subject; determining a protein score based on the protein data; determining whether to obtain genomic data of the sample by comparing the protein score to a threshold; and predicting whether the subject has a condition based on at least one of the protein score or the genomic data.
10. The method of clause 9, wherein the protein data includes measurements of a plurality of proteins of the sample.
11. The method of clause 9 or 10, wherein the protein data is indicative of a predetermined panel of proteins of the sample.
12. The method of clause 11, wherein the predetermined panel includes one or more of CYFRA, CEA, or HE4.
13. The method of clause 11 or 12, wherein the predetermined panel includes one or more of CEA, Ferritin, and anti-p53 antibodies.
14. The method of any of clauses 11-13, wherein the predetermined panel includes one or more of ACE2, ACP5, ACY1, ADA2, ADAM8, ADGRE2, ADGRE5, ADGRG1, AFP, AGR2, ALCAM, ALK, ALPP, ANG, ANG2, ANGPTL4, anti-p53 antibodies, ANXA10, APEX1, AR, AREG, ARSA, B2M, B4GALT1, BAMBI, BMP6, BST2, BTA, BTN2A1, C19ORF12, CA125, CA12, CA15-3, CA19-9, CA242, CA50, CA72.4, CCL14, CCL15, CCL21, CCL22, CD163, CD200, CD200R1, CD22D, CD27, CD274D, CD28, CD300C, CD302, CD38D, CD48, CD6, CD70, CD74, CD79BD, CD83, CDCP1, CDH2, CDH6, CDHR2, CEA, CEACAM21, CEACAM5, CHI3L1, CLEC14A, CLEC5A, CLSTN2, CNDP1, CNTN5, COL4A1D, CRELD2, CRIM1, CRLF1, CRP, CRTAM, CSF1, CTSD, CTSL, CXADR, CXCL13, CXCL17, CXCL9, CYFRA, DCTPP1, DDR1, DKK3, DPP10, DSC2, EFEMP1, EFNA4, EGFL7, ENG, ENPP2, EPHA2D, EPHB4D, EPS8L2, ERBB2D, ESM1, EZR, FABP1, FCRL1, FCRL2, FCRL3, FCRL5, FCRLB, Ferritin, FGFR2B, FLT3LG, FOLR1, FRZB, FSTL3, FUT3, FUT5, FUT5, GAL, GALNT3, Gastrin 17, GCNT1, GDF15, GDNF, GFAP, GGH, GGT1, Glycodelin, GOLM2, GP2, GRN, HAVCR1, HE4, HLA-E, HYOU1, ICAM3, IDS, IFNGR1I, IFNLR1, IGF-1, IGFBP3, IGFBP6, IGFBP7, IGSF3, IL10RB, IL12A, IL12B, IL12BD, IL12RB1, IL18BP, IL18R1, IL2RAB, IL4RD, IL5RAD, IL6R, IL6STD, ITGA5, ITGB2D, ITGB7D, ITIH3, ITM2A, JAM, JCHAIN, KLRB1, KRT14, KRT18, KRT19, LAG3, LAIR2, LAYN, LGALS9, LILRB1, LRIG1, LRP11, LTA, LTBP2, LY9, LYPD8, MARCO, MDK, MILR1, MMED, MMP12, MMP7D, MMP9, MSLN, MSR1, NBL1, NECTIN4D, NFASC, NOMO1, NOS1, NOS3, Notum, NRCAM, NRP2, NT5E, Osteopontin, PARP1, PCDH17, PCOLCE, PDCD1D, PDGFC, Pepsinogen I, Pepsinogen II, PDGFRAB, PGFD, PIGR, PIK3AP1, PIVKA-II, PLAUR, PLXNB2, PREB, PRELP, proGRP, Prolactin, PRSS8, PSA, PSIP1, PTPRS, PVR, RARRES2, RBP2, RBP5, REG3A, REG4, RNASET2, ROR1, RTN4R, SCC, SDC1, SDC4, SEMA4D, SEMA7A, SERPINA9, SEZ6L2, SFTPA2, SH2D1A, SIGLEC1, SIGLEC10, SIGLEC6, SLAMF1, SLAMF6, SLAMF7D, SLAMF8, SPARCL1, SPINK1, SPINK4, SPINT1, SPON2, SPP1, SRP14, ST6GAL1, STC2, STFR2, SULF2, SULT2A1, TCL1A, TFF1, TFF2, TFPI2, TGFBI, TGFBR2, THBD, TIMP1, TNF, TNFRSF10, TNFRSF10A, TNFRSF10B, TNFRSF11B, TNFRSF13, TNFRSF13B, TNFRSF13C, TNFRSF14, TNFRSF19, TNFRSF1A, TNFRSF1B, TNFRSF21, TNFRSF4, TNFRSF8, TNFRSF9, TNFSF13, TNFSF13BD, TNFSF13BI, TNR, TPP1, TREML2, VCAM1, VTCN1, WAS, or YKL40.
15. The method of any of clauses 9-14, further including: receiving proteins obtained from the sample; capturing all or a subset of the proteins; generating detection signals indicative of the captured proteins; detecting, by at least one sensor, the detection signals; and generating the protein data based on the detection signals.
16. The method of clause 15, wherein capturing all or the subset of the proteins includes binding, by a ligand configured to specifically bind to the captured proteins, all or the subset of the proteins.
17. The method of clause 16, wherein the ligand includes at least one of an antibody, an antibody fragment, an aptamer, a protein, a peptide, an enzyme, or a tag.
18. The method of any of clauses 15-17, wherein the detection signals include fluorescent signals, colorimetric signals, absorbance, a refractive index, radioactivity, electrochemical signals, or electrochemiluminescent signals.
19. The method of any of clauses 9-18, further including: receiving the sample.
20. The method of clause 19, wherein the sample includes a tissue biopsy sample, a liquid biopsy sample, or a breath sample.
21. The method of clause 19 or 20, wherein the sample includes a liquid biopsy sample including blood, plasma, cerebrospinal fluid, sputum, stool, urine, lymphatic fluid, or saliva.
22. The method of any of clauses 19-21, wherein the sample is a liquid biopsy sample and includes circulating tumor cells (CTCs).
23. The method of any of clauses 19-22, further including: dividing the sample into a first portion and a second portion; and extracting proteins from the first portion of the sample, and wherein determining whether to obtain the genomic data of the sample includes determining whether to extract nucleic acid molecules from the second portion of the sample.
24. The method of any of clauses 9-23, wherein the subject lacks any apparent disease or other pathological condition.
25. The method of any of clauses 9-24, wherein determining the protein score based on the protein data includes: inputting the protein data into a model configured to determine a probability that the protein data is indicative of the condition; and determining the protein score based on the probability.
26. The method of clause 25, wherein the model includes a machine learning model.
27. The method of clause 26, wherein the machine learning model includes a logistic regression model, a random forest model, an artificial neural network (ANN), a decision tree, a k-nearest neighbor (KNN) model, a support vector machine (SVM), or a naïve Bayes classifier.
28. The method of clause 26 or 27, further including: optimizing parameters of the model based on training data.
29. The method of clause 28, further including: generating the training data by: identifying training protein data associated with samples corresponding to a plurality of individuals omitting the subject; identifying labels indicative of protein scores of the plurality of individuals; and generating the training data based on: the labels; and the training protein data.
30. The method of any of clauses 9-29, wherein the protein score includes one or more numerical values.
31. The method of any of clauses 9-30, wherein the protein score includes a multidimensional output.
32. The method of any of clauses 9-31, wherein determining whether to obtain the genomic data of the sample includes: determining that the protein score is greater than at least one threshold.
33. The method of clause 32, wherein determining whether to obtain the genomic data of the sample further includes: based on comparing the protein score to the at least one threshold, determining that the protein score is indicative of an absence of the condition in the subject to greater than a predetermined certainty, and wherein determining whether to obtain the genomic data of the sample includes determining to refrain from obtaining the genomic data of the sample.
34. The method of clause 32 or 33, wherein determining whether to obtain the genomic data of the sample further includes: based on comparing the protein score to the at least one threshold, determining that the protein score is indicative of a presence of the condition in the subject to greater than a predetermined certainty, and wherein determining whether to obtain the genomic data of the sample includes determining to refrain from obtaining the genomic data of the sample.
35. The method of any of clauses 32-34, wherein determining whether to obtain the genomic data of the sample includes: based on comparing the protein score to the at least one threshold, determining that the protein score is indicative of an absence or presence of the condition in the subject to less than a predetermined certainty; and obtaining the genomic data by sequencing nucleic acid molecules in the sample, wherein predicting whether the subject has the condition based on the protein score is based on the genomic data.
36. The method of clause 35, wherein the genomic data includes results from a DNA methylation test.
37. The method of clause 36, wherein the genomic data of the sample includes results from at least one of: a comprehensive genomic profiling test; a whole genome sequencing (WGS) test; a whole exome sequencing (WES) test; a gene expression profiling test; a cancer hotspot panel test; a DNA fragmentation test; or an RNA fragmentation test.
38. The method of any of clauses 35-37, wherein the genomic data corresponds to a single genomic locus.
39. The method of any of clauses 35-38, wherein the genomic data corresponds to multiple genomic loci.
40. The method of any of clauses 35-39, wherein the nucleic acid molecules include DNA fragments and/or RNA fragments.
41. The method of clause 40, wherein the genomic data indicates at least one of: endpoint counts of the DNA fragments and/or RNA fragments; endpoint positions of the DNA fragments and/or RNA fragments; or lengths of the DNA fragments and/or RNA fragments.
42. The method of clause 40 or 41, wherein the genomic data indicates read depth of the DNA fragments and/or RNA fragments.
43. The method of any of clauses 40-42, wherein the genomic data indicates sequencing data of the DNA fragments and/or RNA fragments.
44. The method of any of clauses 35-43, wherein the genomic data indicates a quantity and/or presence of variants present in the nucleic acid molecules.
45. The method of clause 44, wherein the variants include at least one of a substitution, an insertion, a deletion, a copy number alternation, or a rearrangement of fusion.
46. The method of any of clauses 35-45, wherein obtaining the genomic data by sequencing nucleic acid molecules in the sample includes: extracting the nucleic acid molecules from a portion of the sample.
47. The method of clause 46, wherein the nucleic acid molecules include genomic DNA and/or cDNA.
48. The method of clause 46 or 47, wherein the nucleic acid molecules include one or more of cell-free DNA (cfDNA) and circulating tumor DNA (ctDNA).
49. The method of any of clauses 46-48, wherein the nucleic acid molecules include one or more of messenger RNA, microRNA, or non-coding RNA.
50. The method of any of clauses 35-49, wherein obtaining the genomic data by sequencing the nucleic acid molecules in the sample includes: receiving the nucleic acid molecules obtained from the sample; ligating one or more adapters onto one or more of the nucleic acid molecules from the nucleic acid molecules; amplifying the one or more ligated nucleic acid molecules; capturing all or a subset of the amplified nucleic acid molecules; and sequencing, by a sequencer, the captured nucleic acid molecules to obtain a plurality of sequence reads that represent the captured nucleic acid molecules, thereby generating the genomic data.
51. The method of clause 50, wherein the one or more adapters include amplification primers, flow cell adapter sequences, substrate adapter sequences, or sample index sequences.
52. The method of clause 50 or 51, wherein the captured nucleic acid molecules are captured from the amplified nucleic acid molecules by hybridization to one or more bait molecules.
53. The method of clause 52, wherein the one or more bait molecules include one or more additional nucleic acid molecules, each of the one or more additional nucleic acid molecules including a region that is complementary to a region of a captured nucleic acid molecule.
54. The method of any of clauses 50-53, wherein amplifying the one or more ligated nucleic acid molecules includes performing a polymerase chain reaction (PCR) amplification technique, a non-PCR amplification technique, or an isothermal amplification technique.
55. The method of any of clauses 50-54, wherein sequencing the captured nucleic acid molecules includes use of a massively parallel sequencing (MPS) technique, whole genome sequencing (WGS), whole exome sequencing, targeted sequencing, direct sequencing, or Sanger sequencing.
56. The method of any of clauses 50-55, wherein sequencing the captured nucleic acid molecules includes next-generation sequencing (NGS).
57. The method of any of clauses 50-56, wherein the sequencer includes a next-generation sequencer.
58. The method of any of clauses 50-57, wherein sequencing the captured nucleic acid molecules includes sequencing-by-synthesis or nanopore sequencing.
59. The method of any of clauses 35-58, wherein obtaining the genomic data by sequencing the nucleic acid molecules in the sample includes: generating ligated molecules by ligating adapters onto the nucleic acid molecules; generating amplified ligated molecules by amplifying the ligated molecules; generating, using the amplified ligated molecules, detection signals; detecting, by at least one sensor, the detection signals; and generating the genomic data based on the detection signals.
60. The method of clause 59, wherein the detection signals include electrical signals and/or optical signals.
61. The method of clause 59 or 60, wherein generating, using the amplified ligated molecules, the detection signals includes: synthesizing, by a polymerase using fluorescently tagged nucleotide triphosphates (NTPs), a synthesized nucleic acid molecule that is complementary to one of the amplified ligated molecules, and wherein detecting, by the at least one sensor, the detection signals includes: detecting, by at least one optical sensor, optical signals emitted by the fluorescently tagged NTPs upon binding to the synthesized nucleic acid molecule, the optical signals being indicative of at least one sequence of the nucleic acid molecules of the sample.
62. The method of any of clauses 59-61, wherein generating, using the amplified ligated molecules, the detection signals includes: directing the amplified ligated molecules through a nanopore extending from a first space to a second space through a substrate, and wherein detecting, by the at least one sensor, the detection signals includes: detecting, by sensors disposed in the first space and the second space, an electrical signal over time, the electrical signal being indicative of at least one sequence of the nucleic acid molecules of the sample.
63. The method of any of clauses 59-62, wherein the genomic data indicates a full genome or RNA transcriptome of the sample.
64. The method of any of clauses 59-63, wherein the genomic data indicates a whole exome of the sample.
65. The method of any of clauses 59-64, wherein the genomic data indicates a predetermined panel of genes of the sample.
66. The method of clause 65, wherein the predetermined panel includes one or more of TP53, PTEN, POLE, MKI67, FAT3, TAF1, ZFHX3, RPL22, SPTA1, FAM135B, CSMD3, GIGYF2, CSDE1, MLL4, ATR, CTNNB1, USH2A, LIMCH1, RRN3P2, FBXW7, CDH19, USP9X, COL11A1, BCOR, ARID1A, ZNF770, ARID5B, SLC9A11, KRAS, PNN, INPP4A, CTCF, CHD4, AMY2B, RBMX, PPP2R1A, TNFAIP6, PIK3R1, SGK1, HOXA7, METTL14, HPD, MIR1277, CCND1, MECOM, NFE2L2, or ESR1.
67. The method of clause 65 or 66, wherein the predetermined panel includes one or more of ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, or VEGFB.
68. The method of any of clauses 35-67, further including: determining, based on the genomic data, a genomic score of the subject, wherein predicting whether the subject has the condition is based at least in part on the genomic score.
69. The method of clause 68, wherein the genomic score includes one or more numerical values.
70. The method of clause 68 or 69, wherein the genomic score includes a multidimensional output.
71. The method of any of clauses 68-70, wherein predicting whether the subject has the condition includes: generating input features based on the protein score and/or the genomic score; and predicting, using a predictive model, whether the subject has the condition based on the input features.
72. The method of clause 71, wherein the input features are generated based on the protein score and the genomic score.
73. The method of clause 71 or 72, further including: optimizing parameters of the predictive model based on training data.
74. The method of clause 73, further including: generating the training data by: identifying training protein scores and/or training genomic scores associated with samples corresponding to a plurality of individuals omitting the subject; identifying labels indicative of conditions of the plurality of individuals; and generating the training data based on: the labels; and the training protein scores and/or the training genomic scores.
75. The method of clause 74, wherein optimizing the parameters of the predictive model based on the training data includes: training the predictive model to identify attributes, indicated by the training data, that are predictive of the labels, wherein the input features include instances of the attributes identified via the training of the predictive model.
76. The method of any of clauses 73-75, wherein the predictive model includes at least one machine learning (ML) model.
77. The method of clause 76, wherein the at least one ML model includes at least one of: an artificial neural network (ANN); a logistic regression model; a random forest model; a decision tree; a k-nearest neighbor (KNN) model; a support vector machine (SVM); or a naïve Bayes classifier.
78. The method of clause 76 or 77, wherein optimizing the parameters of the predictive model based on the training data includes: training the at least one ML model based on supervised machine learning, the training data including labels indicating whether population samples are from subjects having the condition.
79. The method of any of clauses 76-78, wherein optimizing the parameters of the predictive model based on the training data includes: training the at least one ML model based on unsupervised machine learning by identifying, based on the training data, a plurality of clusters of the training data.
80. The method of clause 79, further including: identifying at least one cluster, of the plurality of clusters, associated with subjects determined to have the condition, wherein the input features include attributes associated with the at least one cluster.
81. The method of any of clauses 9-80, wherein the condition includes a cancer, a genetic disorder, diabetes, cardiac disease, hypertension, a respiratory disease, a metabolic disorder, an infectious disease, an autoimmune disease, a neurological disorder, or arthritis.
82. The method of clause 81, wherein the cancer is adrenal cancer, bladder cancer, blood cancer, bone cancer, brain cancer, breast cancer, carcinoma, cervical cancer, colon cancer, colorectal cancer, corpus uterine cancer, ear, nose and throat (ENT) cancer, endometrial cancer, esophageal cancer, gastrointestinal cancer, head and neck cancer, Hodgkin's disease, intestinal cancer, kidney cancer, larynx cancer, leukemia, liver cancer, lymph node cancer, lymphoma, lung cancer, melanoma, mesothelioma, myeloma, nasopharynx cancer, a neuroblastoma, non-Hodgkin's lymphoma, oral cancer, ovarian cancer, pancreatic cancer, penile cancer, pharynx cancer, prostate cancer, rectal cancer, sarcoma, seminoma, skin cancer, stomach cancer, a teratoma, testicular cancer, thyroid cancer, uterine cancer, vaginal cancer, a vascular tumor, or combinations or metastases thereof.
83. The method of clause 81 or 82, wherein the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.
84. The method of any of clauses 81-83, wherein the genetic disorder includes Aarskog-Scott syndrome, Aase syndrome, achondroplasia, acrodysostosis, addiction, adreno-leukodystrophy, albinism, ablepharon-macrostomia syndrome, alagille syndrome, alkaptonuria, alpha-1 antitrypsin deficiency, Alport's syndrome, Alzheimer’s disease, amyotrophic lateral sclerosis, asthma, autoimmune polyglandular syndrome, androgen insensitivity syndrome, Angelman syndrome, Apert syndrome, ataxia, ataxia telangiectasia, atherosclerosis, attention deficit hyperactivity disorder (ADHD), autism spectrum disorder, baldness, Batten disease, Becker muscular dystrophy, Beckwith-Wiedemann syndrome, Best disease, bipolar disorder, brachydactyl), Burkitt lymphoma, chronic myeloid leukemia, Charcot-Marie-Tooth disease, Crohn's disease, cleft lip, Cockayne syndrome, Coffin Lowry syndrome, congenital adrenal hyperplasia, Cornelia de Lange syndrome, Costello syndrome, Cowden syndrome, craniofrontonasal dysplasia, Crigler-Najjar syndrome, Creutzfeldt-Jakob disease, cystic fibrosis, deafness, deidocranial dysplasia, depression, diabetes, diastrophic dysplasia, DiGeorge syndrome, Down syndrome, dyslexia, Duchenne muscular dystrophy, Dubowitz syndrome, ectodermal dysplasia, Ellis-van Creveld syndrome, Ehlers-Danlos, epidermolysis bullosa, epilepsy, essential tremor, Fabry disease, facial scapulohumeral dystrophy, familial hypercholesterolemia, familial Mediterranean fever, fragile X syndrome, Friedreich's ataxia, Gaucher disease, glaucoma, glucose galactose malabsorption, glutaricaciduria, gyrate atrophy, Goldberg Shprintzen syndrome, Gorlin syndrome, Hailey-Hailey disease, Haw River syndrome, hemihypertrophy, hemochromatosis, hemophilia, hereditary motor and sensory neuropathy (HMSN), an HLA-B27-associated disorder, Huntington's disease, hypertrophic cardiomyopathy, immunodeficiency with hyper-IgM, Jacobsen syndrome, juvenile onset diabetes, Kennedy’s disease, Klinefelter's syndrome, Kabuki syndrome, Leigh's disease, limb genital syndrome, long QT syndrome, manic depression, Marfan syndrome, Menkes syndrome, miscarriage, mucopolysaccharide disease, multiple endocrine neoplasia, multiple sclerosis, muscular dystrophy, myoclonus epilepsy, myotonic dystrophy, neurofibromatosis, Niemann-Pick disease, Noonan syndrome, obesity, occipital epilepsy syndrome, Parkinson disease, paroxysmal nocturnal hemoglobinuria, Pendred syndrome, peroneal muscular atrophy, phenylketonuria (PKU), polycystic kidney disease, polycystic ovary syndrome, Prader-Willi syndrome, primary biliary cirrhosis, REAR syndrome, Refsum disease, retinitis pigmentosa, retinoblastoma, Rett syndrome, Sanfilippo syndrome, schizophrenia, severe combined immunodeficiency, sickle cell anemia, spina bifida, spinal muscular atrophy, spinocerebellar atrophy, a SRY-associated disorder, sudden adult death syndrome, Tangier disease, Tay-Sachs disease, thalasseaemia, thrombocytopenia absent radius syndrome, Townes-Brocks syndrome, tuberous sclerosis, Turner syndrome, Usher syndrome, von Hippel-Lindau syndrome, Von Willebrand disease, Waardenburg syndrome, Weaver syndrome, Werner syndrome, Williams syndrome, Wilson's disease, xeroderma pigmentosum, or Zellweger syndrome.
85. The method of any of clauses 81-84, wherein the diabetes includes type 1 diabetes, type 2 diabetes, gestational diabetes mellitus (GDM), monogenic diabetes, or secondary diabetes.
86. The method of any of clauses 81-85, wherein the cardiac disease includes angina, pericarditis, stroke, coronary artery disease, heart valve disease, left ventricular heart failure, right ventricular heart failure, systolic heart failure, diastolic heart failure, ischemic heart disease, or an arrhythmia.
87. The method of any of clauses 81-86, wherein the hypertension includes primary hypertension, secondary hypertension, or pulmonary hypertension.
88. The method of any of clauses 81-87, wherein the respiratory disease includes asthma, cystic fibrosis, bronchitis, pleural effusion, pneumonia, bronchiectasis, chronic obstructive pulmonary disease, idiopathic pulmonary fibrosis, acute lung injury, acute respiratory distress syndrome, emphysema, or bronchiolitis obliterans.
89. The method of any of clauses 81-88, wherein the metabolic disorder includes osteoporosis, hypothyroidism, hyperthyroidism, a mitochondrial disorder, type 2 diabetes, metabolic syndrome, non-alcoholic fatty liver disease, metabolic dysfunction-associated steatotic liver disease (MASLD), alcoholic ketoacidosis, Cushing’s syndrome, hypercalcemia, or porphyria.
90. The method of any of clauses 81-89, wherein the infectious disease includes a viral disease, a bacterial disease, a parasitic disease, a fungal disease, or a prion disease.
91. The method of clause 90, wherein the viral disease includes or is caused by hepatitis C, norovirus, junin virus, dengue virus, coronavirus, human immunodeficiency virus, herpes simplex, avian flu, chickenpox, cold sores, common cold, glandular fever, influenza, measles, mumps, pharyngitis, pneumonia, rubella, severe acute respiratory syndrome, respiratory syncytial virus, dengue virus, chikungunya virus, lassa virus, ebolavirus, nipah virus, varicella zoster virus, cytomegalovirus (CMV), Epstein-Barr virus (EBV), encephalitis, poliovirus, coxsackievirus, an enterovirus, LaCrosse virus, or rabies virus.
92. The method of clause 90 or 91, wherein the bacterial disease includes a disease caused by E. coli, E. coli STEC, Neisseria gonorrhoeae, Neisseria meningitides, Salmonella enteritidis, Streptococcus pneumoniae, Streptococcus pyogenes, Haemophilus influenza, Klebsiella pneumoniae, Pseudomonas aeruginosa, Staphylococcus aureus, Vibrio cholera, Brucella suis, Mycobacterium tuberculosis, Enterobacter cloacae, Bacillus cereus, Corynebacterium diphtheria, Campylobacter jejuni, Listeria monocytogenes, Campylobacter coli, Bacillus thuringiensis, Yersinia enterocolitica, Helicobacter pylori, Bacteroides fragilis, Clostridium butyricum, Streptococcus agalactiae, Leuconostoc lactis, Shigella sonnei, Zymomonas mobilis, Treponema denticola, Lactobacillus plantarum, Enterococcus faecium, Bacillus liquefaciens, Staphylococcus epidermidis, Serratia marcescens, Citrobacter freundii, Parvimonas micra, Prevotella intermedia, Brucella melitensis, Aeromonas caviae, Clostridium botulinum, Clostridium perfringens, Mycoplasma pneumonia, Salmonella typhimurium, Salmonella paratyphi, Shigella flexnerii, Vibrio parahaemolyticus, Yersinia pseudotuberculosis, Actinobacter baumanii, Bacteroides distasonis, Clostridium leptum, Brevibacterium lactofermentum, Actinobacillus actinobycetemcomitans, Selnomonas ruminatium, Mycoplasma mycoides, Staphylococcus lugdunensis, Lactobacillus rhamnosus, Lactobacillus casei, Lactobacillus acidophilus, Bacillus coagulans, Pyrococcus abyssi, Selenomonas nominantium, Streptococcus fetus, Streptomyces phaechromogenes, Streptomyces ghanaenis, Enterobacter aerogenes, Morganella morganii, Fusobacterium nucleatum, Porphyromonas endodontalis, Porphyromonas gingivalis Nicrococcus luteus, Aeromonas hydrophila, Bacillus anthracis, Bartonella henselae, Bordetella pertussis, Borrelia garinii, Borrelia burgdorferi, Brucella canis, Campylobacter fetus, Clostridium tetani, Francisella tularensis, Legionella pneumophila, Leptospira interrogans, Leptospira santarosai, Leptospira weilii, Mycobacterium leprae, Shigella dysenteriae, Staphylococcus saprophyticus, Streptococcus viridans, Treponema pallidum, Ureaplasma urealyticum, Yersinia pestis, or Propionibacterium acnes.
93. The method of any of clauses 90-92, wherein the fungal disease is caused by Aspergillus, Bipolaris, Blastomyces, Candida, Cryptococcus, Coccidioides, Curvularia, Exophiala, Histoplasma, Mucorales, Ochroconis, Pseudallescheria, Ramichloridium, Sporothrix, Zygomyctes, Pneumocystis, or Trichosporon.
94. The method of any of clauses 90-93, wherein the parasitic disease includes or is caused by Acanthamoeba keratitis, Amoebiasis, Ascariasis, Babesiosis, Balantidiasis, Baylisascariasis, Chagas disease, Clonorchiasis, Cochliomyia, Cryptosporidiosis, Diphyllobothriasis, Dracunculiasis, Echinococcosis, Elephantiasis, Enterobiasis, Fascioliasis, Fasciolopsiasis, Filariasis, Giardiasis, Gnathostomiasis, Hymenolepiasis, Isosporiasis, Katayama fever, Leishmaniasis, Lyme disease, Malaria, Metagonimiasis, Myiasis, Onchocerciasis, Pediculosis, Scabies, Schistosomiasis, Sleeping sickness, Strongyloidiasis, Taeniasis, Toxocariasis, Toxoplasmosis, Trichinosis, Trichuriasis, or trypanosomiasis.
95. The method of any of clauses 90-94, wherein the prion disease includes at least one of: Creutzfeldt-Jakob disease (CJD), Fatal familial insomnia, Kuru, or Gerstmann-Strassler-Scheinker Disease.
96. The method of any of clauses 81-95, wherein the autoimmune disease includes atopic disease, paraneoplastic autoimmune diseases, arthritis, rheumatoid arthritis, juvenile ankylosing spondylitis, juvenile Reiter's Syndrome, seronegativity, enthesopathy, arthropathy (SEA) Syndrome, dermatomyositis, scleroderma, lupus, systemic lupus erythematosus (SLE), vasculitis, ankylosing spondylitis, Reiter's Syndrome, myolitis, polymyolitis, dermatomyolitis, polyarteritis nodossa, Wegener's granulomatosis, polymyalgia rheumatica, sarcoidosis, sclerosis, primary biliary sclerosis, sclerosing cholangitis, Sjogren's syndrome, psoriasis, plaque psoriasis, guttate psoriasis, inverse psoriasis, pustular psoriasis, erythrodermic psoriasis, dermatitis, atopic dermatitis, atherosclerosis, Still's disease, myasthenia gravis, inflammatory bowel disease (IBD), Crohn's disease, ulcerative colitis, celiac disease, multiple sclerosis (MS), asthma, COPD, rhinosinusitis, eosinophilic esophagitis, eosinophilic bronchitis, Guillain-Barre disease, Type I diabetes mellitus, thyroiditis, Addison's disease, Raynaud's phenomenon, autoimmune hepatitis, graft-versus-host disease (GVHD), transplantation rejection, autoimmune kidney disease, pernicious anemia, alopecia areata, Graves’ disease, Hashimoto Thyroiditis, vitiligo, or inflammation.
97. The method of any of clauses 81-96, wherein the neurological disorder includes dementia, Alzheimer’s disease, vascular dementia, Lewy body dementia, frontotemporal dementia, Parkinson’s disease, multiple sclerosis (MS), epilepsy, stroke, migraine, amyotrophic lateral sclerosis (ALS), Huntington’s disease, Guillain-Barré syndrome (GBS), myasthenia gravis, Bell’s palsy, cerebral palsy, neurofibromatosis, myalgic encephalomyelitis, autism spectrum disorder, attention deficit hyperactivity disorder (ADHD), chronic inflammatory demyelinating polyneuropathy (CIDP), or muscular dystrophy.
98. The method of any of clauses 81-97, wherein the arthritis includes osteoarthritis, rheumatoid arthritis, psoriatic arthritis, gout, ankylosing spondylitis, juvenile arthritis, septic arthritis, or Still's disease.
99. The method of any of clauses 9-98, wherein predicting whether the subject has the condition includes: predicting whether the subject has a type of the condition; predicting whether the subject has a subtype of the condition; predicting a metastasis profile of the subject; predicting a survivability of the subject; predicting a symptom of the subject; predicting an effective therapy to treat the condition; predicting a resistance of the subject to a treatment of the condition; a predicted stage of the condition; a predicted grade of the condition; or a predicted Eastern Cooperative Oncology Group (ECOG) performance status of the subject.
100. The method of any of clauses 9-99, further including: based on predicting whether the subject has the condition, identifying a suggested treatment for the subject.
101. The method of clause 100, wherein the suggested treatment includes administration of one or more therapeutic agents predicted to treat the condition of the subject.
102. The method of clause 101, further including: administering the one or more therapeutic agents to the subject.
103. The method of any of clauses 100-102, wherein the suggested treatment includes at least one of chemotherapy, radiation therapy, immunotherapy, a targeted therapy, or surgery.
104. The method of any of clauses 9-103, further including: identifying one or more risk factors for the condition, wherein predicting whether the subject has the condition is further based on the one or more risk factors.
105. The method of clause 104, wherein the one or more risk factors include at least one of: a family history of the condition; lifestyle factors associated with the condition; or demographic information of the subject.
106. The method of any of clauses 9-105, further including: based on predicting whether the subject has the condition, determining whether to perform a follow-up diagnostic test.
107. The method of clause 106, further including performing the follow-up diagnostic test.
108. The method of clause 107, wherein the follow-up diagnostic test includes at least one of a physical exam, biopsy, a sequencing-based test, diagnostic imaging, or a histological study.
109. The method of clause 108, wherein the sequencing-based test includes whole transcriptome sequencing, cfRNA sequencing, whole exome sequencing, whole genome sequencing, a cancer hotspot panel test, a DNA fragmentation test, an RNA fragmentation test, a microsatellite instability (MSI) test, or a tumor mutational burden (TMB) test.
110. The method of clause 108 or 109, wherein the diagnostic imaging includes at least one of magnetic resonance imaging, a computed tomography scan, ultrasound, X-ray, a mammogram, positron emission tomography, bone scintigraphy, myelography, a colonoscopy, echocardiography, radiography, nuclear medicine, fluoroscopy, or single-photon emission computed tomography.
111. The method of any of clauses 9-110, further including: generating a report indicating whether the subject is predicted to have the condition; and outputting the report.
112. The method of clause 111, wherein outputting the report includes: transmitting data indicating the report to an external device.
113. The method of clause 112, wherein the external device is associated with the subject and/or a healthcare provider.
114. The method of clause 112 or 113, wherein the data is transmitted over one or more communication networks.
115. The method of any of clauses 112-114, wherein the data is transmitted over a peer-to-peer connection.
116. The method of any of clauses 112-115, wherein outputting the report includes: visually presenting, by a display, the report.
117. The method of any of clauses 112-116, further including: based on predicting whether the subject has the condition, determining one or more therapies to treat the condition of the subject, wherein the report further indicates the one or more therapies.
118. The method of clause 117, wherein the one or more therapies includes a dosage of one or more therapeutic agents predicted to treat the condition of the subject.
119. The method of any of clauses 9-118, further including: based on predicting whether the subject has the condition, determining whether the subject is eligible for a clinical trial.
120. A system, including: at least one processor; and memory storing instructions that, when executed by the at least one processor, cause the at least one processor to perform operations including: identifying protein data of a sample obtained from a subject; determining a protein score based on the protein data; determining whether to obtain genomic data of the sample by comparing the protein score to a threshold; and predicting whether the subject has a condition based on at least one of the protein score or the genomic data.
121. The system of clause 120, further including: a protein detector configured to generate the protein data by detecting a plurality of proteins in the sample.
122. The system of clause 120 or 121, further including: a sequencer configured to generate the genomic data by sequencing a plurality of nucleic acid molecules in the sample.
123. The system of any of clauses 120-122, further including: a transceiver configured to transmit data indicating at least one of: the protein score, the genomic data of the subject, or an indication of whether the subject is predicted to have the condition.
124. The system of any of clauses 120-123, further including: an output device configured to output an indication of at least one of: the protein score, the genomic data of the subject, or an indication of whether the subject is predicted to have the condition.
125. A non-transitory computer readable medium storing instructions for performing operations including: identifying protein data of a sample obtained from a subject; determining a protein score based on the protein data; determining whether to obtain genomic data of the sample by comparing the protein score to a threshold; and predicting whether the subject has a condition based on at least one of the protein score or the genomic data.
126. A method, including: receiving, by one or more processors, a first data based on a first set of nucleic acid molecules from a liquid biopsy sample obtained from a subject; determining, by the one or more processors, a first score based on the first data; determining, by the one or more processors, that the first score is greater than a threshold; and based on determining that the first score is greater than the threshold: receiving, at the one or more processors, sequence read data for a second data based on a second set of nucleic acid molecules from the liquid biopsy sample; generating, by the one or more processors, input features based on the first data and the second data; and determining, using a classifier executed by the one or more processors, a likelihood that the subject has at least one type of cancer based on the input features.
127. The method of clause 126, wherein the first data is indicative of methylation of the first set of the nucleic acid molecules.
128. The method of clause 126 or 127, wherein the nucleic acid molecules include cfDNA and/or ctDNA.
129. The method of clause 128, further including: extracting, from the liquid biopsy sample, the cfDNA and/or the ctDNA.
130. The method of any of clauses 126-129, wherein the first data corresponds to a single genomic locus.
131. The method of any of clauses 126-130, wherein the first data corresponds to multiple genomic loci.
132. The method of any of clauses 126-131, wherein the first data corresponds to a predetermined panel of genes including at least one of: CYFRA, CEA, and HE4.
133. The method of any of clauses 126-132, wherein the first data corresponds to a predetermined panel of genes including at least one of: CEA, Ferritin, and anti-p53 antibodies.
134. The method of any of clauses 126-133, further including: sequencing the second set of the nucleic acid molecules using NGS, a MPS technique, WGS, whole exome sequencing, targeted sequencing, direct sequencing, or Sanger sequencing.

### Conclusion

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e., denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the,” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

Tumor mutational burden (TMB) is a measure of the number of mutations carried by tumor cells. By comparing DNA sequences from a patient’s healthy tissues and tumor cells, the number of acquired somatic mutations present in tumors, but not in normal tissues, may be determined. In some instances, driver mutations may be excluded from a TMB calculation.

In certain examples, "tumor mutational burden" or “TMB” refers to the number of somatic mutations in a tumor's genome and/or the number of somatic mutations per area of the tumor's genome. In some embodiments, TMB, as used herein, refers to the number of somatic mutations per megabase (Mb) of DNA sequenced. In some embodiments, germline (inherited) variants are excluded when determining TMB, given that the immune system has a higher likelihood of recognizing these as self. In various cases, driver mutations are excluded from a TMB calculation.

Microsatellites are highly polymorphic DNA-repeat regions. In certain examples, “microsatellite” refers to a repetitive nucleic acid having repeat units of less than about 10 base pairs or nucleotides in length. In certain examples, a microsatellite refers to a tract of tandemly repeated (i.e., adjacent) DNA motifs ranging from one to six or up to ten nucleotides, with each motif repeated 5 to 50 repeated times. “Microsatellite instability” refers to genetic instability in the microsatellite regions. Cancer patients with microsatellite instability classified as being high (MSI-H or MSI-High) frequently exhibit an accumulation of somatic mutations in tumor cells that leads to a range of molecular and biological changes including high tumor mutational burden, increased expression of neoantigens and abundant tumor-infiltrating lymphocytes. Chang et al. “Microsatellite Instability: A Predictive Biomarker for Cancer Immunotherapy,” Appl Immunohistochem Mol Morphol, 26(2):e15-e21 (2018). These changes have been linked to increased sensitivity to checkpoint inhibitor drugs, such as pembrolizumab, which is used to treat advanced melanoma, head and neck squamous cell carcinoma, non-small cell lung cancer (NSCLC), and classical Hodgkin lymphoma.

A viral status test refers to a test that identifies the presence of viral RNA or DNA in a subject. The test can identify viral load and/or viral identity. For example, the viral status test can identify the presence of viral RNA or DNA associated with the occurrence of certain cancers. Examples of such viruses include Hepatitis B Virus (HBV) and Hepatitis C Virus (HCV), Kaposi Sarcoma-Associated Herpesvirus (KSHV), Merkel Cell Polyomavirus (MCV), Human Papillomavirus (HPV), Human Immunodeficiency Virus Type 1 (HIV-1, or HIV), Human T-Cell Lymphotropic Virus Type 1 (HTLV-1), and Epstein-Barr Virus (EBV).

Cancer “hotspot” mutations give rise to oncological outcomes. PhyloP, SIFT, Grantham, COSMIC and PolyPhen-2 are in silico tools that can be used to assess pathogenicity of identified variants. Exemplary hotspot genes and mutations include EGFR exon 19 activating mutation, EGFR exon 19 deletion, EGFR exon 19 insertion, EGFR exon 19 sensitizing mutation, EGFR exon 20 activation mutation, EGFR exon 20 insertion, EGFR G719 mutation, EGFR L858R mutation, EGFR L861 mutation, EGFR S768 mutation, EGFR T790M mutation, C797 mutation, KIT activating mutation, KRAS activating mutation, MET activating mutation, NRAS activating mutation, PMS2 promoter mutations, among many others. Hotspot mutations also occur in the following genes: AKT2, BRCA1, BRCA2, ERC1, NSD1, POLH, PPM1G, PTEN, RAD18, RAD51, RAD51B, RB1, TERT, TP53, TP53Bp1, ALK, ARMT1, ATAD5, ATG7, ATIC, AXL, BIRC6, BRD3, BRD4, CAPRIN1, CCAR2, CCDC6, CDK5RAP2, CHD9, CIT, CTNNB1, CUL1, EBF1, EIF3E, HIP1, HMGA2, IRF2BP2, NOTCH1, NOTCH4, NPM1, OFD1, TACC1, TACC3, TERF2, TMEM106B, UBE2L3, USP10, WRDR48, YAP1, ZEB2, and ZMYND8.

A “DNA methylation test” refers to an assay, which can be commercially available, for distinguishing methylated versus unmethylated cytosine loci in DNA. Techniques for measuring cytosine methylation include bisulfite-based methylation assays. The addition of bisulfite to DNA results in the methylation of unmethylated cytosine and its ultimate conversion to the nucleotide uracil. Uracil has similar binding properties to thiamine in the DNA sequence. Previously methylated cytosine does not undergo similar chemical conversion on exposure to bisulfite. Bisulfite assays can thus be used to discriminate previously methylated versus unmethylated cytosine.

An exemplary quantitative methylation detection assay combines bisulfite treatment and restriction analysis COBRA, which uses methylation sensitive restriction endonucleases, gel electrophoresis, and detection based on labeled hybridization probes. (Ziong and Laird, Nucleic Acid Res. 1997 25; 2532-4). Another exemplary detection assay is the methylation specific polymerase chain reaction PCR (MSPCR) for amplification of DNA segments of interest. This assay can be performed after sodium bisulfite conversion of cytosine and uses methylation sensitive probes. Other detection assays include the Quantitative Methylation (QM) assay, which combines PCR amplification with fluorescent probes designed to bind to putative methylation sites; MethyLight^{TM} (Qiagen, Redwood City, CA) a quantitative methylation detection assay that uses fluorescence-based PCR (Eads, et al., Cancer Res. 1999; 59:2302-2306); and Ms-SNuPE, a quantitative technique for determining differences in methylation levels in CpG sites. As with other techniques, Ms-SNuPE also requires bisulfite treatment to be performed first, leading to the conversion of unmethylated cytosine to uracil while methyl cytosine is unaffected. PCR primers specific for bisulfite converted DNA are then used to amplify the target sequence of interest. The amplified PCR product is isolated and used to quantitate the methylation status of the CpG site of interest. (Gonzalgo and Jones Nuclei Acids Res1997; 25:252-31).

In particular embodiments, pyrosequencing can be used to detect marker methylation. Pyrosequencing is a method of DNA sequencing that relies on detection of the release of pyrophosphates as DNA is synthesized (and is therefore a “sequencing by synthesis” technique). To assess methylation by pyrosequencing, a DNA sample can be incubated with sodium bisulfite, converting unmethylated cytosine to uracil. The presence of uracil will result in thymine incorporation during PCR amplification. Therefore, sequencing results that include thymine at a nucleotide position that is known to encode cytosine can be interpreted as unmethylated sites. In contrast cytosines present in the sequencing results indicate that the site was methylated in the original DNA sample, because methylation protects cytosine from conversion to uracil upon treatment. Bisulfite treatment can also be performed on control samples with known methylation patterns, to reduce or eliminate false positive results. Commercially available pyrosequencing machines include Pyro Mark Q96 (Qiagen, Hilden, Germany). For more details on methods to use pyrosequencing for measurement of methylation, see Delaney et al. Methods Mol Biol. 2015 1343: 249-264. Pyrosequencing is especially useful for detecting methylation in the CpG sites within genes.

In particular embodiments, a protein marker is detected by contacting a sample with reagents (e.g., antibodies), generating complexes of reagent and marker(s), and detecting the complexes. Particular embodiments for detecting and measuring protein levels can use methods including agglutination, chemiluminescence, electro-chemiluminescence (ECL), enzyme-linked immunoassays (ELISA), immunoassay, immunoblotting, immunodiffusion, immunoelectrophoresis, immunofluorescence, immunohistochemistry, immunoprecipitation, mass-spectrometry, and western blot. See also, e.g., E. Maggio, Enzyme-Immunoassay (1980), CRC Press, Inc., Boca Raton, Fla; and U.S. Pat. Nos. 4,727,022; 4,659,678; 4,376,110; 4,275,149; 4,233,402; and 4,230,797.

Read depth refers to the number of times that a specific genomic site is sequenced during a sequencing run.

Certain implementations are described herein, including the best mode known to the inventors for carrying out implementations of the disclosure. Of course, variations on these described implementations will become apparent to those of ordinary skill in the art upon reading the foregoing description. The inventor expects skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

What is claimed is:

1. A method, comprising:
dividing a liquid biopsy sample obtained from a subject into a first portion and a second portion;
providing a plurality of proteins obtained from the first portion of the liquid biopsy sample;
detecting the plurality of proteins, thereby generating protein data;
generating a first score based on the protein data;
determining that the first score is greater than a threshold; and
based on determining that the first score is greater than the threshold:
providing a plurality of nucleic acid molecules obtained from the second portion of the liquid biopsy sample;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, all or a subset of the captured amplified nucleic acid molecules to obtain a plurality of sequence reads that represent the sequenced amplified nucleic acid molecules, thereby generating sequence read data;
receiving, at one or more processors, the sequence read data for the plurality of sequence reads;
generating, by the one or more processors, input features based on at least one of the protein data and the sequence read data; and
determining, using a classifier executed by the one or more processors, a likelihood that the subject has at least one type of cancer based on the input features.

2. The method of claim 1, wherein the plurality of proteins comprise at least one of CYFRA, CEA, or HE4.

3. A method, comprising:
identifying protein data of a sample obtained from a subject;
determining a protein score based on the protein data;
determining whether to obtain genomic data of the sample by comparing the protein score to a threshold; and
predicting whether the subject has a condition based on at least one of the protein score or the genomic data.

4. The method of claim 3, wherein the protein data comprises measurements of a plurality of proteins of the sample, and/or
wherein the protein data is indicative of a predetermined panel of proteins of the sample.

5. The method of claim 4, wherein the predetermined panel comprises one or more of CEA, Ferritin, and anti-p53 antibodies.

6. The method of claim 3, wherein the sample comprises a tissue biopsy sample, a liquid biopsy sample, or a breath sample.

7. The method of claim 3, wherein determining the protein score based on the protein data comprises:
inputting the protein data into a model configured to determine a probability that the protein data is indicative of the condition; and
determining the protein score based on the probability.

8. The method of claim 7, wherein the model comprises a machine learning model, the method further comprising:
optimizing parameters of the model based on training data.

9. The method of claim 3, wherein determining whether to obtain the genomic data of the sample further comprises:
comparing the protein score to at least one threshold; and
based on comparing the protein score to the at least one threshold, determining that a probability that the protein data is indicative of an absence of the condition in the subject is greater than a predetermined probability, and
wherein determining whether to obtain the genomic data of the sample comprises determining to refrain from obtaining the genomic data of the sample.

10. The method of claim 3, wherein determining whether to obtain the genomic data of the sample further comprises:
comparing the protein score to at least one threshold; and
based on comparing the protein score to the at least one threshold, determining that a probability that the protein data is indicative of a presence of the condition in the subject is greater than a predetermined probability, and
wherein determining whether to obtain the genomic data of the sample comprises determining to refrain from obtaining the genomic data of the sample.

11. The method of claim 3, wherein determining whether to obtain the genomic data of the sample comprises:
comparing the protein score to at least one threshold;
based on comparing the protein score to the at least one threshold, determining that a probability that the protein data is indicative of an absence or presence of the condition in the subject to less than a predetermined probability; and
obtaining the genomic data by sequencing nucleic acid molecules in the sample,
wherein predicting whether the subject has the condition based on the protein score is based on the genomic data.

12. The method of claim 11, wherein the genomic data of the sample comprises results from at least one of:
a DNA methylation test;
a comprehensive genomic profiling test;
a whole genome sequencing (WGS) test;
a whole exome sequencing (WES) test;
a gene expression profiling test;
a cancer hotspot panel test;
a DNA fragmentation test; or
an RNA fragmentation test.

13. The method of claim 11, wherein the nucleic acid molecules comprise DNA fragments and/or RNA fragments; wherein the genomic data indicates at least one of:
endpoint counts of the DNA fragments and/or RNA fragments;
endpoint positions of the DNA fragments and/or RNA fragments; or
lengths of the DNA fragments and/or RNA fragments;
read depth of the DNA fragments and/or RNA fragments; or
sequencing data of the DNA fragments and/or RNA fragments.

14. The method of claim 3, wherein the genomic data indicates a full genome or RNA transcriptome of the sample, or
wherein the genomic data indicates a predetermined panel of genes of the sample.

15. The method of claim 3, wherein predicting whether the subject has the condition comprises:
generating input features based on the protein score and/or the genomic score; and
predicting, using a predictive model, whether the subject has the condition based on the input features.

16. The method of claim 15, wherein the predictive model comprises at least one machine learning (ML) model, the method further comprising:
generating training data by:
identifying training protein scores and/or training genomic scores associated with samples corresponding to a plurality of individuals omitting the subject;
identifying labels indicative of conditions of the plurality of individuals; and
generating the training data based on:
the labels; and
the training protein scores and/or the training genomic scores; and
optimizing parameters of the predictive model based on the training data.

17. The method of claim 3, wherein the condition comprises a cancer, a genetic disorder, diabetes, cardiac disease, hypertension, a respiratory disease, a metabolic disorder, an infectious disease, an autoimmune disease, a neurological disorder, or arthritis.

18. The method of claim 3, further comprising:
based on predicting whether the subject has the condition, identifying a suggested treatment for the subject, the suggested treatment comprising administration of one or more therapeutic agents predicted to treat the condition of the subject; and
administering the one or more therapeutic agents to the subject.

19. A system, comprising:
at least one processor; and
memory storing instructions that, when executed by the at least one processor, cause the at least one processor to perform operations comprising:
identifying protein data of a sample obtained from a subject;
determining a protein score based on the protein data;
determining whether to obtain genomic data of the sample by comparing the protein score to a threshold; and
predicting whether the subject has a condition based on at least one of the protein score or the genomic data.

20. The system of claim 19, further comprising one or more of:
a protein detector configured to generate the protein data by detecting a plurality of proteins in the sample;
a transceiver configured to transmit data indicating at least one of: the protein score, the genomic data of the subject, or an indication of whether the subject is predicted to have the condition; or
an output device configured to output an indication of at least one of: the protein score, the genomic data of the subject, or an indication of whether the subject is predicted to have the condition.
